(19) United States
(12) Patent Application Publication
(10) Pub. No.: US 2019/0013094 A1
(43) Pub. Date: Jan. 10, 2019

(54) CANCER CARE NAVIGATION METHODS

(71) Applicant: Frances Mary Johnson, Houston, TX (US)

(72) Inventor: Frances Mary Johnson, Houston, TX (US)

(21) Appl. No.: 15/911,590

(22) Filed: Mar. 5, 2018

Related U.S. Application Data
(60) Provisional application No. 62/529,841, filed on Jul. 7, 2017.

(51) Int. Cl. G16H 40/20; G16H 10/20; G16H 10/60

(52) U.S. Cl. CPC G16H 40/20; G06Q 50/22; G16H 10/60; G16H 10/20

(57) ABSTRACT

One or more specific versions disclosed herein include a method in a computing system for adjusting a role of a cancer care navigator that includes: receiving patient data relating to an evaluation by a patient of one or more navigation steps of cancer care delivered by a navigator to the patient; retrieving from a database data relating to evaluations by other patients of one or more navigation steps of cancer care delivered by the navigator to the other patients; aggregating the retrieved data stored in the memory; assessing some of the received patient data including comparing some of the received patient data with some of the aggregated retrieved data to provide an evaluation data assessment; formulating an adjustment proposal to adjust the role of the navigator in delivering one or more navigation steps based at least in part on the evaluation data assessment; and communicating the adjustment proposal.

### BACKGROUND

### 1. Field of Inventions

The field of this application and any resulting patent relates to cancer care, primarily navigation programs for cancer care.

### 2. Description of Related Art

The Institute of Medicine (IOM, 2013) has concluded that the cancer care delivery system is in crisis due to a growing demand for cancer care and a shrinking workforce. One of the typical problems with cancer care delivery programs is that they are often uncoordinated and not patient-centered. Patient navigation programs have been identified as one potential solution (Moy & Chabner, 2011). Generally speaking, patient navigation in cancer care “refers to individualized assistance offered to patients, families, and caregivers to help overcome health care system barriers and facilitate timely access to quality medical and psychosocial care from pre-diagnosis through all phases of the cancer experience.” (See C-Change, 2005, p. 1). The American College of Surgeons Commission on Cancer (ACSCOC) requires that a navigation process be in place in order for cancer programs to receive accreditation (ACSCOC, 2014).

One of the features of navigation programs is the involvement of nurse practitioner (NP) navigators, who have been shown to help in the delivery of more cost-effective quality care, saving millions of dollars (American Nurses Association, 2012). Oncology NP navigators are those nurse practitioners having a certification in oncology and who utilize navigation processes to care for cancer patients along any part of the cancer care continuum, from intake through survivorship or end-of-life care.

A systematic literature review was conducted to ascertain current knowledge related to oncology nurse practitioner navigation, with complete findings published elsewhere (Johnson, 2015). One study (Jean-Pierre et al., 2011) described a preliminary framework for the navigation process, and found that patient outcomes were influenced by patients, navigators, navigation process, and external factors. The Donabedian model (1966) has stressed the critical linkage between the role that processes have in determining outcomes, and the challenges that are involved in determining cause and effects of these organizational components. Although few systematic studies define standardized outcome measurements for nurse practitioners in the oncology setting (Johnson, 2015), certain consortiums have defined those outcome measurements on a global basis (Battaglia, Burhansstipanov, Murrell, Dwyer, & Caron, 2011).

Various methods and systems have been proposed and utilized for patient navigation in the cancer care arena, including certain methods and systems disclosed in some of the references appearing on the face of this patent. However, each of those methods and systems lack the combination of steps and/or features of the methods, systems, and/or computer-readable media (“CRM”) covered by the patent claims below. Furthermore, it is contemplated that the methods, systems, and computer-readable media covered by at least some of the claims of this issued patent solve at least some of the problems that certain prior art methods and systems have failed to solve. Also, the methods, systems, and computer-readable media covered by at least some of the claims of this patent have benefits that would be surprising and unexpected to a person of ordinary skill in the art based on the prior art existing as of the filing date of this application.

### SUMMARY

Certain embodiments disclosed herein include methods in a computing system having one or more programmable processors communicatively coupled to memory and a database for adjusting a role of a cancer care navigator that include: receiving patient data relating to an evaluation by a patient of one or more navigation steps of cancer care delivered by a navigator to the patient, wherein the navigation steps are selected from the group consisting of one or more barrier assessments, triaging, resourcing, and guidance performed for the patient; storing the received patient data in the memory; retrieving from the database data relating to evaluations by other patients of one or more navigation steps of cancer care delivered by the navigator to the other patients; storing the retrieved data in the memory; aggregating the retrieved data stored in the memory; assessing some of the received patient data including comparing some of the received patient data with some of the aggregated retrieved data to provide an evaluation data assessment; formulating an adjustment proposal to adjust the role of the navigator in delivering one or more navigation steps based at least in part on the evaluation data assessment; and communicating the adjustment proposal, wherein the role of the navigator is subsequently adjusted in delivering one or more navigation steps based on some portion of the communicated adjustment proposal.

Certain embodiments disclosed herein include methods in a computing system having one or more programmable processors communicatively coupled to memory and a database for adjusting a role of a cancer care navigator that include: receiving patient data relating to an evaluation by a patient of one or more navigation steps of cancer care delivered by a navigator to the patient; storing the received patient data in the memory; receiving navigator data relating to an evaluation by the navigator of the one or more navigation steps of cancer care delivered by the navigator to the patient; storing the received navigator data in the memory; assessing some of the received patient data including comparing some of the received patient data with some of the received navigator data to provide an evaluation data assessment; formulating an adjustment proposal to adjust the role of the navigator in delivering one or more navigation steps based at least in part on the evaluation data assessment; communicating the adjustment proposal, wherein the role of the navigator is subsequently adjusted in delivering one or more navigation steps based on some portion of the communicated adjustment proposal; and wherein the navigation steps are selected from the group consisting of one or more barrier assessments, triaging, resourcing, and guidance performed for the patient.

Certain embodiments disclosed herein include cancer care navigation methods that include: (a) receiving electronically transmitted evaluation data for a patient undergoing cancer care, wherein the evaluation data correspond to one or more navigation steps performed by a particular cancer care navigator for that patient wherein the navigation steps are selected from the group consisting of patient-level, facility-level, or community-level barrier assessments, triage, resourcing, and guidance performed for that patient; (b) assessing at least some of the received electronically transmitted evaluation data including comparing at least some of the received electronically transmitted evaluation data with electronically stored evaluation data corresponding to other patients to provide an evaluation data assessment; (c) formulating an adjustment proposal relating to one or more of the navigation steps corresponding to at least some of the assessed evaluation data wherein the adjustment proposal is based at least in part on the evaluation data assessment; and (d) communicating the adjustment proposal, wherein the role of the cancer care navigator is subsequently adjusted based on some portion of the communicated adjustment proposal. The systems and CRM disclosed herein preferably include instructions capable of performing one or more of the aforementioned steps (a) through (d).

Certain embodiments disclosed herein also include cancer care navigation methods that include: (a) receiving electronically transmitted evaluation data for a patient undergoing cancer care, wherein the evaluation data correspond to one or more navigation steps performed for that patient selected from the group consisting of patient-level, facility-level, or community-level barrier assessments, triage, resourcing, or guidance performed for that patient; (b) assessing at least some of the transmitted electronically transmitted evaluation data; (c) formulating an adjustment proposal relating to one or more of the navigation steps selected from the group consisting of patient-level, facility-level, or community-level barrier assessments, triage, resourcing, and guidance, based at least in part on some portion of the assessment of the electronically transmitted evaluation data; and (d) communicating the adjustment proposal to an administrator of the patient-level, facility-level, or community-level barrier assessments, triage, resourcing, or guidance, wherein (i) any adjustment proposal regarding delivery of patient-level, barrier assessments, triage, resourcing, or guidance is communicated to a patient-level administrator, (ii) any adjustment proposal regarding delivery of facility-level barrier assessments, triage, resourcing, or guidance is communicated to a facility-level administrator, and (iii) any adjustment proposal regarding delivery of community-level barrier assessments, triage, resourcing, or guidance is communicated to a community-level administrator. The systems and CRM disclosed herein preferably include instructions capable of performing one or more of the aforementioned steps (a) through (d).

### BRIEF DESCRIPTION OF THE DRAWINGS

FIG. 1 is a schematic diagram of a system that includes a network in which client computers and a central server computer are interconnected, as described more fully below.

FIG. 2 depicts a representation of elements and steps of certain versions of systems and methods disclosed herein.

FIG. 3 is a flowchart showing at least one specific embodiment of disclosed methods.

FIG. 4A is an illustration of certain evaluation algorithms.

FIG. 4B depicts a screen-shot of a display showing visual comparison of evaluation data.

### DETAILED DESCRIPTION

### I. Introduction

A detailed description will now be provided. The purpose of this detailed description, which includes the drawings, is to satisfy the statutory requirements of 35 U.S.C. § 112. For example, the detailed description includes a description of the inventions and sufficient information that would enable a person having ordinary skill in the art to make and use the inventions defined by the claims. In the figures, like elements are generally indicated by like reference numerals regardless of the view or figure in which the elements appear. The figures are intended to assist the description and to provide a visual representation of certain aspects of the subject matter described herein. The figures are not all necessarily drawn to scale, nor do they show all the structural details of the systems, nor do they limit the scope of the claims.

Each of the appended claims defines a separate invention which, for infringement purposes, is recognized as including equivalents of the various elements or limitations specified in the claims. Depending on the context, all references below to the “invention” may in some cases refer to certain specific embodiments only. In other cases, it will be recognized that references to the “invention” will refer to the subject matter recited in one or more, but not necessarily all, of the claims. Each of the inventions will now be described in greater detail below, including specific embodiments, versions, and examples, but the inventions are not limited to these specific embodiments, versions, or examples, which are included to enable a person having ordinary skill in the art to make and use the inventions when the information in this patent is combined with available information and technology. Various terms as used herein are defined below, and the definitions should be adopted when construing the claims that include those terms, except to the extent a different meaning is given within the specification or in express representations to the Patent and Trademark Office (PTO). To the extent a term used in a claim is not defined below or in representations to the PTO, it should be given the broadest definition persons having skill in the art have given that term as reflected in at least one printed publication, dictionary, or issued patent.

### 2. Certain Specific Embodiments

Below, certain specific embodiments of methods, systems, and CRM are described, which are by no means an exclusive description of the inventions. Other specific embodiments, including those referenced in the drawings, are encompassed by this application and any patent that issues therefrom.

Certain embodiments disclosed herein include methods in a computing system having one or more programmable processors communicatively coupled to memory and a database for adjusting a role of a cancer care navigator that include: receiving patient data relating to an evaluation by a patient of one or more navigation steps of cancer care delivered by a navigator to the patient, wherein the navigation steps are selected from the group consisting of one or more barrier assessments, triaging, resourcing, and guidance performed for the patient; storing the received patient data in the memory; retrieving from the database data relating to evaluations by other patients of one or more navigation steps of cancer care delivered by the navigator to the other patients; storing the retrieved data in the memory; aggregating the retrieved data stored in the memory; assessing some of the received patient data including comparing some of the received patient data with some of the aggregated retrieved data to provide an evaluation data assessment; formulating an adjustment proposal to adjust the role of the navigator in delivering one or more navigation steps based at least in part on the evaluation data assessment; and communicating the adjustment proposal, wherein the role of the navigator is subsequently adjusted in delivering one or more navigation steps based on some portion of the communicated adjustment proposal.

Certain embodiments disclosed herein include methods in a computing system having one or more programmable processors communicatively coupled to memory and a database for adjusting a role of a cancer care navigator that include: receiving patient data relating to an evaluation by a patient of one or more navigation steps of cancer care delivered by a navigator to the patient; storing the received patient data in the memory; receiving navigator data relating to an evaluation by the navigator of the one or more navigation steps of cancer care delivered by the navigator to the patient; storing the received navigator data in the memory; assessing some of the received patient data including comparing some of the received patient data with some of the received navigator data to provide an evaluation data assessment; formulating an adjustment proposal to adjust the role of the navigator in delivering one or more navigation steps based at least in part on the evaluation data assessment; communicating the adjustment proposal, wherein the role of the navigator is subsequently adjusted in delivering one or more navigation steps based on some portion of the communicated adjustment proposal; and wherein the navigation steps are selected from the group consisting of one or more barrier assessments, triaging, resourcing, and guidance performed for the patient.

Certain embodiments disclosed herein include cancer care navigation methods that include: (a) receiving electronically transmitted evaluation data for a patient undergoing cancer care, wherein the evaluation data correspond to one or more navigation steps performed by a particular cancer care navigator for that patient wherein the navigation steps are selected from the group consisting of patient-level, facility-level, or community-level barrier assessments, triage, resourcing, and guidance performed for that patient; (b) assessing at least some of the received electronically transmitted evaluation data including comparing at least some of the received electronically transmitted evaluation data with electronically stored evaluation data corresponding to other patients to provide an evaluation data assessment; (c) formulating an adjustment proposal relating to one or more of the navigation steps corresponding to at least some of the assessed evaluation data wherein the adjustment proposal is based at least in part on the evaluation data assessment; and (d) communicating the adjustment proposal, wherein the role of the cancer care navigator is subsequently adjusted based on some portion of the communicated adjustment proposal. The systems and CRM disclosed herein preferably include instructions capable of performing one or more of the aforementioned steps (a) through (d).

Certain embodiments disclosed herein also include cancer care navigation methods that include: (a) receiving electronically transmitted evaluation data for a patient undergoing cancer care, wherein the evaluation data correspond to one or more navigation steps performed for that patient selected from the group consisting of patient-level, facility-level, or community-level barrier assessments, triage, resourcing, or guidance performed for that patient; (b) assessing at least some of the transmitted electronically transmitted evaluation data; (c) formulating an adjustment proposal relating to one or more of the navigation steps selected from the group consisting of patient-level, facility-level, or community-level barrier assessments, triage, resourcing, and guidance, based at least in part on some portion of the assessment of the electronically transmitted evaluation data; and (d) communicating the adjustment proposal to an administrator of the patient-level, facility-level, or community-level barrier assessments, triage, resourcing, or guidance, wherein (i) any adjustment proposal regarding delivery of patient-level, barrier assessments, triage, resourcing, or guidance is communicated to a patient-level administrator, (ii) any adjustment proposal regarding delivery of facility-level barrier assessments, triage, resourcing, or guidance is communicated to a facility-level administrator, and (iii) any adjustment proposal regarding delivery of community-level barrier assessments, triage, resourcing, or guidance is communicated to a community-level administrator. The systems and CRM disclosed herein preferably include instructions capable of performing one or more of the aforementioned steps (a) through (d).

In any of the methods, systems, or CRM disclosed herein the evaluation data assessment may be communicated to the cancer care navigator or to an administrator of the patient-level, facility-level, or community-level barrier assessments, triage, resourcing, or guidance; and the formulating of the adjustment proposal may be performed by the cancer care navigator or the administrator of the patient-level, facility-level, or community-level barrier assessments, triage, resourcing, or guidance.

In any of the methods, systems, or CRM disclosed herein the evaluation data may correspond to how the patient or the cancer navigator subjectively views the one or more navigation steps performed by the particular cancer care navigator for that patient.

In any of the methods, systems, or CRM disclosed herein the evaluation data may have a number of forms, e.g., the data can be binary corresponding to satisfactory (e.g., “good”) or unsatisfactory (e.g., “bad”) or the evaluation data can be a numerical answer to a proposition corresponding to the navigation step.

In any of the methods, systems, or CRM disclosed herein the evaluation data may include numbers selected from 1, 2, 3, 4, or 5, in which 1 means “strongly agree,” 2 means “agree,” 3 means “no opinion or neutral,” 4 means “disagree” and 5 means “strongly disagree.”

In any of the methods, systems, or CRM disclosed herein communicating the adjustment proposal may include electronically transmitting the adjustment proposal to the cancer care navigator or to an administrator of the patient-level, facility-level, or community-level barrier assessments, triage, resourcing, or guidance.

Any of the methods disclosed herein may additionally include the step of adjusting the role of the cancer care navigator.

In any of the methods disclosed herein the adjusting of the role of the cancer care navigator may include: (a) adding new tasks for the cancer care navigator; (b) modifying existing tasks performed by the cancer care navigator; (c) supplying information to the cancer care navigator relating to any of the navigation steps or to the adjustment proposal; or (d) making recommendations to the cancer care navigator.

In any of the methods disclosed herein: (i) any adjustment proposal regarding delivery of patient-level, barrier assessments, triage, resourcing, or guidance can be communicated to a patient-level administrator, (ii) any adjustment proposal regarding delivery of facility-level barrier assessments, triage, resourcing, or guidance can be communicated to a facility-level administrator, and (iii) any adjustment proposal regarding delivery of community-level barrier assessments, triage, resourcing, or guidance can be communicated to a community-level administrator.

In any of the methods, systems, or CRM disclosed herein the evaluation data may be electronically transmitted from a computer operated by the patient after or while the patient undergoes one or more of the navigation steps.

In any of the methods, systems, or CRM disclosed herein the evaluation data may be electronically transmitted from a computer operated by the patient's navigator after or while the patient undergoes one or more of the navigation steps.

In any of the methods, systems, or CRM disclosed herein the evaluation data can be received by a server that is connected to multiple client computers that may be each capable of electronically transmitting evaluation data for multiple patients undergoing cancer care in the same facility, wherein the evaluation data for each of the multiple patients preferably corresponds to the same navigation steps albeit for different patients.

In any of the methods, systems, or CRM disclosed herein the evaluation data may correspond to navigation steps that may include patient-level, facility-level, and community-level barrier assessments, triage, resourcing, or guidance performed for that particular patient.

In any of the methods, systems, or CRM disclosed herein the assessing of at least some of the received electronically transmitted evaluation data may include formulating metrics corresponding to the evaluation data.

In any of the methods, systems, or CRM disclosed herein the comparing of at least some of the received electronically transmitted evaluation data with electronically stored evaluation data corresponding to other patients may be selected from the group consisting of (a) comparing evaluation data corresponding to a first navigator with evaluation data corresponding to other navigators in the same or different facilities; (b) comparing evaluation data with a predetermined benchmark; and (c) comparing evaluation data corresponding to a first navigator cancer care for a first patient with evaluation data corresponding to the first navigator for cancer care for one or more other patients.

Any of the methods, systems, or CRM disclosed herein may additionally include displaying on a computer screen an image that includes a visual representation of at least some of the received electronically transmitted evaluation data. In certain specific embodiments, the image may additionally include a visual representation of electronically stored evaluation data corresponding to other patients. In certain specific embodiments, the visual representation of evaluation data may include an image of a bar chart, which may include one set of bars corresponding to evaluation data for the patient and another set of bars corresponding to other patients.

Any of the methods, systems, or CRM disclosed herein may additionally include storing the received patient data to the database.

In any of the methods, systems, or CRM disclosed herein the received patient data may additionally include one or more values identifying an evaluation by the patient of the one or more navigation steps of cancer care delivered by the navigator to the patient.

In any of the methods, systems, or CRM disclosed herein the received navigator data may additionally include one or more values identifying an evaluation by the navigator of the one or more navigation steps of cancer care delivered by the navigator to the patient.

Any of the methods, systems, or CRM disclosed herein may additionally include the step of assessing of at least some of the received patient data, which step includes: calculating a difference between some of the received patient data and some of the aggregated retrieved data; and determining whether the difference equals or exceeds a predetermined threshold.

Any of the methods, systems, or CRM disclosed herein may additionally include: formulating a patient metric based on at least some of the received patient data; formulating a benchmark based on at least some of the aggregated retrieved data; and assessing the patient metric with the benchmark to provide an evaluation data assessment.

Any of the methods, systems, or CRM disclosed herein may additionally include the step of assessing of the patient metric with the benchmark, which step includes: calculating a difference between the metric based on at least some of the received patient data and the benchmark based on at least some of the aggregated retrieved data; and calculating an excess value between the difference and a predetermined threshold.

Any of the methods, systems, or CRM disclosed herein may additionally include: formulating a patient metric based on at least some of the received patient data; formulating a navigator metric based on at least some of the received navigator data; and assessing the patient metric based on the received patient data with the navigator metric based on the received navigator data to provide an evaluation data assessment.

Any of the methods, systems, or CRM disclosed herein may additionally include the step of assessing of the patient metric with the navigator metric, which step includes: calculating a difference between some of the received patient data and some of the received navigator data; and calculating an excess value between the difference and a predetermined threshold.

### 3. Specific Embodiments in the Figures

The drawings presented herein are for illustrative purposes only do not limit the scope of the claims. Rather, the drawings are intended to help enable one having ordinary skill in the art to make and use the claimed inventions.

Although the systems and processes described herein have been described in detail, it should be understood that various changes, substitutions, and alterations can be made without departing from the spirit and scope of the invention as defined by the following claims. Those skilled in the art may be able to study the preferred embodiments and identify other ways to practice the invention that are not exactly as described herein. It is the intent of the inventors that variations and equivalents of the invention are within the scope of the claims, and the description, abstract and drawings are not to be used to limit the scope of the invention. The invention is specifically intended to be as broad as the claims below and their equivalents.

This section addresses specific embodiments of the invention shown in the drawings, which relate to methods, systems, and CRM for adjusting one or more roles of a cancer care navigator to improve a navigation program for cancer treatment. Although this discussion focuses on the drawings, and the specific embodiments referenced therein, the discussion may also have applicability to other embodiments not shown in the drawings, e.g. other types of treatment besides cancer care. However, the limitations referenced in this section should not be used to limit the scope of the claims themselves, which have broader applicability.

One of the goals of the analysis of the evaluation data was improvement in the navigation process itself. Often, the failure to expedite care along the “cancer continuum” can result in treatment delays and patients being “stuck in the system.” As explained more fully elsewhere herein, the navigator (“N”) is typically trained to expedite care by conducting barrier-focused assessments, triaging, pulling in resources, and guiding the patient (“P”) to the next step in the cancer care process. In accordance with certain specific embodiments (e.g., methods, systems and/or CRM) described herein, the analyses of some or all of the evaluation data identified herein is designed to provide for specific tracking and navigation program improvement. The navigator should be a center for care not only for the patient but also within the facility and community, thus, the navigator's role preferably extends beyond the traditional nurse-patient relationship. The navigator can be involved in this navigation process itself within the facility and community. By continuing to act as an interface between the patient, facility, and community, and by receiving evaluation data from the patient, the navigator is able to function as a center for care for all those involved in the patient's cancer journey.

As mentioned above, one of the overall goals for a navigator is to expedite patient care. Another goal is to provide high-quality care to the patient efficiently and in a reasonably prompt manner, without undue delays, and at a reasonable cost. Recognizing that terms like “high-quality,” “efficiently,” “reasonably prompt,” “undue delays,” and “reasonable cost” are relative and even subjective, one of the goals of the methods and systems described herein is to provide targeted improvements to a particular cancer care program that a particular patient is experiencing based on data that are accurate and measurable, and in some cases objective (e.g., certain metrics), either data from that particular patient's own cancer care or data from the cancer care for previous patients, or both. In certain specific embodiments, the improvement takes place after the patient has already finished the particular program, in which case other patients may experience the benefits of an improved program. In other specific embodiments, the patient will experience benefits of an improved program while the patient is still participating in the program, for example, where evaluation data is analyzed and specific adjustments are made to the downstream portions of that particular cancer care program.

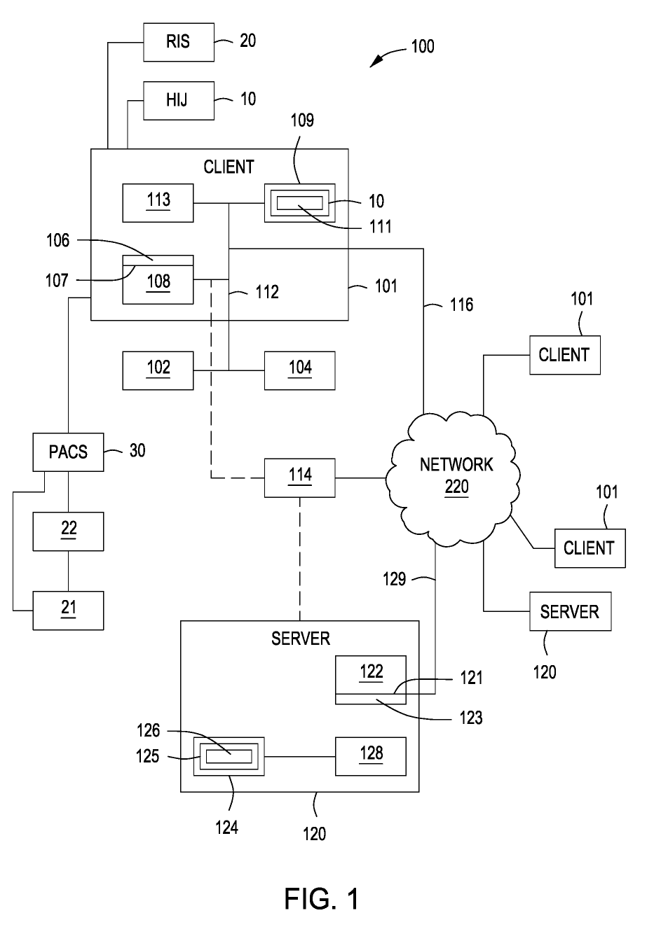

As discussed elsewhere herein, at least one example of a navigation tool is a client computer, which can include or be used to access a user interface which can then be displayed for immediate use by the patient or navigator. FIG. 1 is a schematic diagram of a system that includes a network in which client computers and a central server computer are interconnected. The computer hardware and system connectivity of FIG. 1 are illustrative and conventional, and various other combinations of system components can be used to carry out the methods herein for entering and transmitting evaluation data. An example of a conventional system that can be used for entering and transmitting the evaluation data described herein can be found in U.S. Patent Publication No. US 2014/0358585, which includes a schematic diagram identified therein as FIG. 1, which schematic diagram is hereby incorporated by reference. In addition, a description of the various parts of the schematic diagram of FIG. 1 can be found in the '585 publication, specifically in paragraphs [0039] through [0067] of that publication, and the contents of those paragraphs are also incorporated herein by reference, except that the descriptions of the data transmitted in the '585 publication are not incorporated by reference, given that the nature of the evaluation data of the present application and resulting patent is different from the nature of the data being transmitted and processed in the '585 publication. For convenience and ease of reference, the present application and resulting patent includes a schematic diagram (FIG. 1 herein) with a substantially different appearance but essentially the same components and numbering scheme as the schematic diagram of the '585 publication.

The processing described below may be performed by a single system or by a distributed processing computer system. In addition, such processing and functionality can be implemented in the form of special purpose hardware or in the form of software or firmware being run by a general purpose or network processor. Data handled in such processing or created as a result of such processing can be stored in any type of memory as is conventional in the art. By way of example, such data may be stored in a temporary memory, such as in the RAM of a given computer system or subsystem. In addition, or in the alternative, such data may be stored in longer term storage devices, such as magnetic disks, rewritable optical disks, and so on. For purposes of the disclosure herein, a computer-readable media (CRM) may include any form of data storage mechanism, including existing memory technologies as well as hardware or circuit representations of such structures and of such data.

The techniques of the present system and method might be implemented using a variety of technologies. For example, the methods described herein may be implemented in software running on a programmable processor, or implemented in hardware utilizing either a combination of microprocessors or other specially designed application specific integrated circuits, programmable logic devices, or various combinations thereof. In particular, the methods described herein may be implemented by a series of computer-executable instructions residing on a storage medium such as a carrier wave, disk drive, or other computer-readable medium.

The system may be operated online, via the Internet, as a web-based platform and accessible to users, e.g., patients, health care navigators and administrators, or anyone stakeholder authorized to access the system.

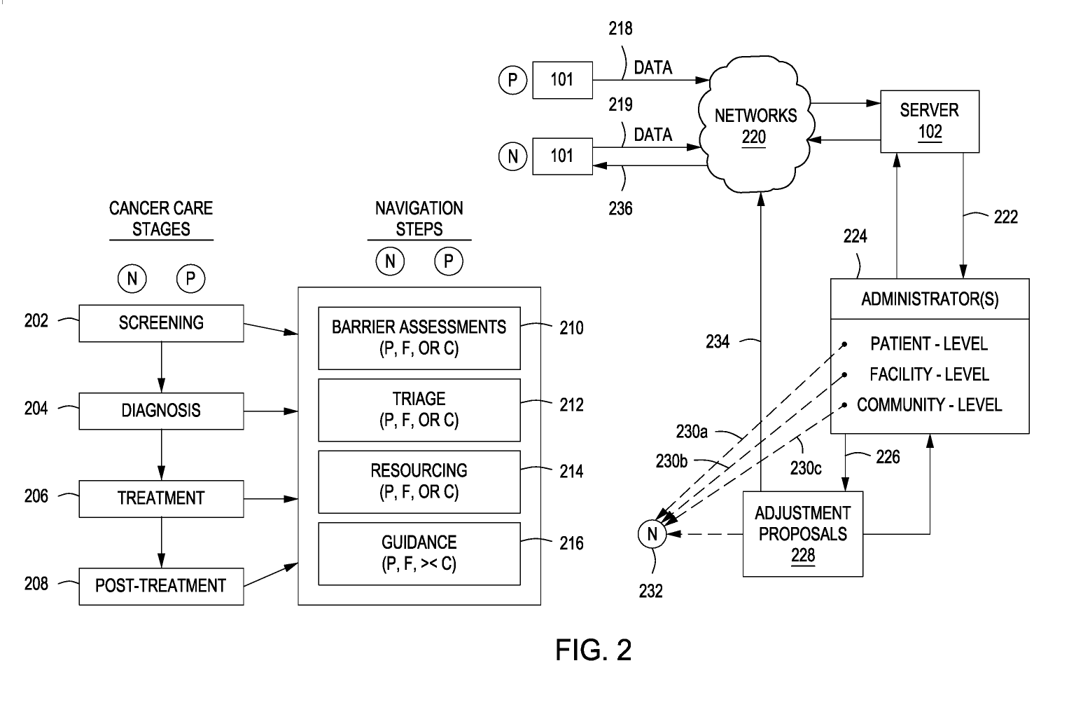

One or more specific embodiments of the methods disclosed herein are depicted in FIG. 2, which are more fully discussed below. Some steps or aspects of methods for evaluating health care provided to cancer patients in a health care facility may include providing health care to a patient, e.g., providing cancer care to a patient. The cancer care stages depicted in FIG. 2 include screening 202, diagnosis 204, treatment 206, and post-treatment 208. Also depicted in FIG. 2 are certain navigation steps, which may include 210 assessing barriers to care, followed by triaging 212, resourcing 214, and guidance 216, e.g., guiding the patient to the patient's next step(s) in the cancer care process. Each of those navigation steps can be classified further as either a patient-level (P), facility-level (F), or community-level (C) navigation step. Those navigation steps may then be evaluated, in the form of “evaluation data,” discussed below and elsewhere herein.

During or after the process of cancer care for a patient, the patient may be requested to share his or her evaluation of the care provided to the patient during each navigation step. Additionally, a navigator for the patient may be requested to share his or her evaluation of the care provided to the patient during each navigation step.

As shown in FIG. 2, individual client computers 101 can be operated by a navigator N and/or a patient P to record his or her evaluation. The client computer 101 may provide a user interface having a set of questions to which a user, e.g., navigator or patient, may enter an evaluation response for each question. Each response by the user may be stored as evaluation data in memory on the client computer 101 for later transmission. After the evaluation data is entered it can be either transmitted 218 through wires or wirelessly from the client computer 101 operated by the patient or transmitted 219 from the client computer 101 operated by the navigator. The evaluation data may be transmitted to a network 220 and thence to a server 102, e.g., a central server which may be any computer or combination of computers that are programmed and have the necessary functionality by which the evaluation data can be assessed, e.g., analyzed, as discussed elsewhere herein.

Once the transmitted evaluation data is received by the server 102, one or more processors on the server 102 may read the evaluation data. Additionally, the one or more processors may instruct the system 100 to store the evaluation data to memory. Furthermore, the one or more processors may instruct the system 100 to store the evaluation data on a database on a storage device 128 of the server 102. Afterwards, the one or more processors may operate to assess the transmitted evaluation data. Based at least in part on one or more portions of the evaluation data assessment, the one or more processors may operate to generate one or more adjustment proposals 228 that include the evaluation data assessment, e.g. comparisons. Additionally, proposals may include patient-level proposals, facility level-proposals, and community-level proposals.

As exemplified in FIG. 2, the adjustment proposals 228 can then be provided, e.g., transmitted 222 by the server 102, to one or more administrators 224, which preferably include patient-level administrator(s), facility-level administrator(s), and/or community-level administrators. The proposals are preferably transmitted electronically, e.g. email, to respective administrators 224, who may then communicate the proposals 230a, 230b, and/or 230c to the navigator 232. Alternatively, once an administrator receives the adjustment proposals, the administrator may discuss the adjustment proposals directly with the navigator 232. The adjustment proposal may also be transmitted electronically 234 to the network, where it can be then transmitted 236 to the client computer 101 operated by the navigator.

Since the transmitted patient evaluation data and/or navigator evaluation data may be stored to the database, the evaluation data may be accessed from the database later to re-execute any of the disclosed steps on the server, e.g., assessment of the evaluation data.

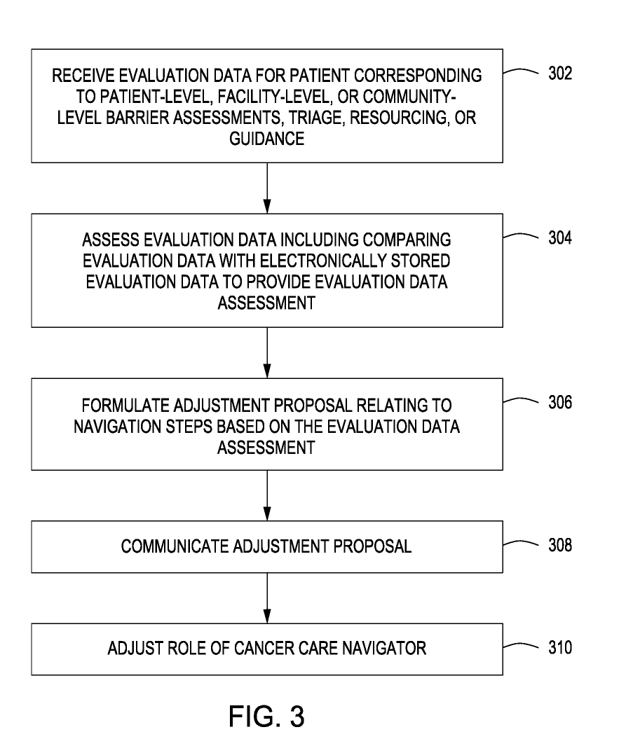

Referring now to FIG. 3, a flow chart depicts examples of how evaluation data for a patient, corresponding to patient-level, facility-level, or community-level barrier assessments, triage, resourcing, and guidance performed for that patient may be received 302 by the system 100 using the one or more programmable processors, and then at least some of the received electronically transmitted evaluation data may be assessed 304 by the system 100, e.g., by comparing at least some of the received electronically transmitted evaluation data with electronically stored evaluation data to provide an evaluation data assessment. Then an adjustment proposal may be formulated 306 by the system 100 relating to one or more of the navigation steps corresponding to at least some of the assessed evaluation data. Next, the adjustment proposal may be communicated 308 by the system 100; and the role of the cancer care navigator 310 may be adjusted based on some portion of the communicated adjustment proposal.

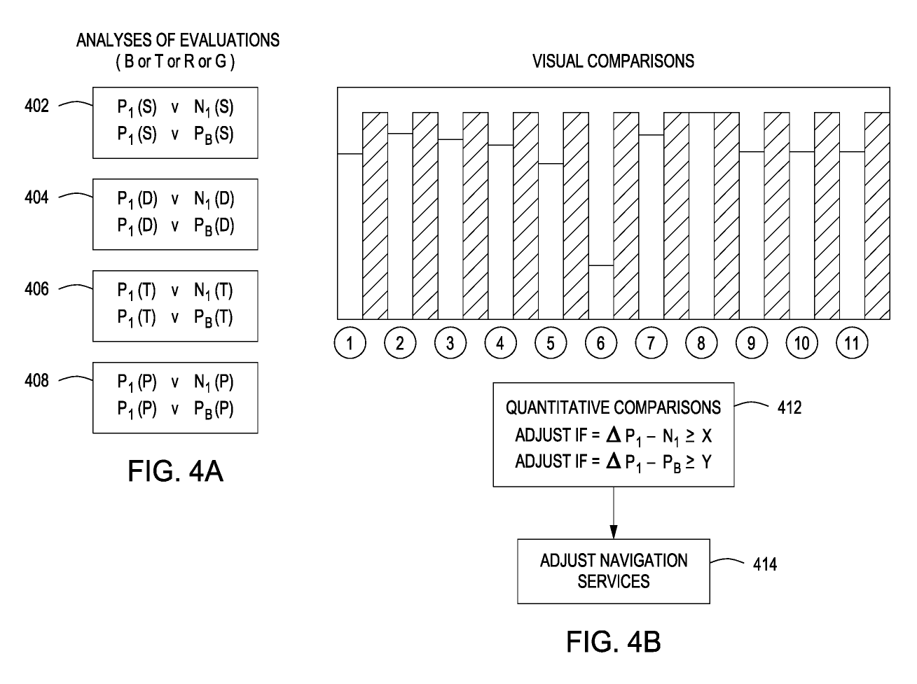

As noted above, the evaluation data received from the navigator and/or patient can be assessed, e.g., analyzed, by the system 100 using the one or more programmable processors. Referring now to FIG. 4A, the analyses may include a screening-stage comparison 402, which is P1(S) versus N1(S), which refers to the comparison between the evaluation data transmitted by the patient for which the navigator is performing navigation services at the screening stage with the evaluation data transmitted by the navigator performing those navigation services for that patient. The screening stage comparison 402 may also be P1(S) versus PB(S), which refers to the comparison between the evaluation data transmitted by the patient for which the navigator is performing navigation services at the screening stage with the evaluation data transmitted by the some benchmark (B) for evaluation data provided by other patients, e.g., an average of data for patients handled by that particular navigator or an average of data for patients in the same facility, or some other statistical aggregation of patient evaluation data.

The analyses may also include diagnosis-stage comparison 404, such as P1(D) versus N1(D), which can be the same comparison as the P1(S) versus N1(S), except it refers to the evaluation of navigation steps at the diagnostic stage rather than the screening stage. Similarly, P1(D) versus PB (D) can be the same comparison as the P1(S) versus PB(S), except it refers to the evaluation of navigation steps at the diagnostic stage rather than the screening stage. Similarly, treatment stage comparisons 406 may include P1(T) versus N1(T) and PP1(T) versus PB(T) comparisons; and post-treatment stage comparisons 408 may include P1(P) versus N1(P) and P1(P) versus PB( ) comparisons.

FIG. 4B shows an example of a visual comparison of certain evaluation data, depicted here as a screen-shot showing a bar chart 410. In this example, there are eleven pairs of bars, with the left-side bar for each pair representing a patient's answer in the form of a number 1, 2, 3, 4, or 5 that is an answer to a propositional statement discussed elsewhere herein identifying an evaluation of a navigation step, e.g., whether the patient was satisfied with information provided by the navigator about insurance coverage, e.g., an example of patient-level barrier-assessment. The right-side cross-hatched bar for that same pair represents the navigator's answer in the form of a number 1, 2, 3, 4, or 5 that is an answer to the same propositional statement answered by the patient. Advantageously, the side-by-side bar-chart comparison for individual propositions provides an administrator with a quick and easy way to identify any problem areas, based on the patterns. For example, although nearly all of the “scores” by the patient are lower than those of the navigator, the difference between the score of the patient and that of the navigator is particularly high for item number 6, suggesting a problem area that needs attention, particularly if this same pattern is repeated for an extended period of time and for many different patients, and even more so when the same difference (delta) is not present for other navigators. As an alternative, or in addition to visual comparisons 410, another analysis is a quantitative comparison 412, in which a predetermined threshold X or Y indicates a delta that is excessive and thus a need for adjusting the role of the navigator in a particular respect. As noted in FIG. 4B, the results of any comparison whether visual or quantitative, may lead to the adjusting of the navigation services, e.g., the role of the navigator in some respect.

In general, the evaluation data can include one or more reports of good or bad levels of health care for the cancer patient, or for a plurality of cancer patients. That evaluation data preferably include evaluations one or more of the navigation steps. e.g., of barrier assessment, triaging, resourcing, and guiding. The evaluation data preferably also include metrics related to the cancer care delivered, such as measurements of the time delays between the various points in the cancer care process such as the time from when diagnostic tests are ordered until the delivery of the test results. As described in greater detail below, the evaluation data preferably includes numerical answers to questions, and those answers preferably are prepared by the patient and the patient's navigator, e.g., the nurse practitioner responsible for the patient. Then, all of some or the evaluation data (e.g., answers), are transmitted electronically to a central server by the patient from a computer held or controlled by the patient, and other answers to the same questions are also transmitted electronically to a server, e.g., the same server, by the navigator from a computer held or controlled by the navigator.

Preferably, any of the evaluation data corresponds to how the patient and/or navigator subjectively view a particular aspect of the delivered health care, e.g. the cancer care. Preferably, the aspect of health care that is being evaluated is some aspect of the navigation itself, e.g., one or more of the navigation steps, rather than merely an aspect of the medical treatment, e.g., the accuracy of the diagnosis or the effectiveness of the treatment. An evaluation can be as simple as “good” or “bad.” Alternatively, it can be a more nuanced numerical evaluation. The term “numerical evaluation” as used herein is defined as a numerical answer to some proposition involving the evaluated health care aspect, e.g., barrier assessment, triaging, resourcing, and guidance. For example, in response to the general proposition “I am satisfied with the information I received from my navigator for the following topics,” the numerical answers can be a number selected from a range of numbers, e.g., 1, 2, 3, 4, or 5. Those numbers represent a scale of 1 to 5 where 1 means “strongly agree,” 2 means “agree,” 3 means “no opinion or neutral,” 4 means “disagree” and 5 means “strongly disagree.” Examples of propositions for selected health care aspects of a cancer care program are shown in Tables 4-9, and a numerical evaluation, e.g., an evaluation of 1, 2, 3, 4, or 5, can be matched to each proposition and then transmitted from a client computer to a server, where the evaluations can be assessed. Based on those assessments, improvements can be made to the cancer care program.

Any of the methods described herein may also include receiving the transmitted evaluation data, and then assessing the evaluation data, e.g., using a central server that is programmed to make such assessments. The step of assessing at least some of the evaluation data can generally include identifying clusters of good or bad levels of health care. Additionally, the assessed evaluation data can be used to adjust the cancer care provided to that particular patient or alternatively to adjust the cancer care provided to future patients.

As discussed elsewhere herein, certain navigation steps can be performed as part of a cancer care continuum. For example, as described below, a navigator performs navigation steps involving the patient and the navigator, which may include barrier assessments and triage, providing of resources, and guiding the patient to the next step. Those navigation steps can then be evaluated during the navigation process itself, using navigation tools such as the systems which will be described in greater detail below. Thus, at least one feature of certain methods, systems, and CRM described herein is a “navigation tool.” A navigation tool can be a guide for tracking as well as for process development thus tying the patient to the overall system. One type of navigation tool is a “patient tool,” which is any tool used for a specific patient, e.g., a “client computer” as discussed elsewhere herein. Note that to the extent the client computer is connected to a server that is also connected to other client computers in the facility, the client computer is also a “facility tool.” In addition to the client computers described below, other navigation tools may be used, preferably in combination with the client computers and software in which the transmitted evaluation data described herein. Examples of those other patient tools include chart review, templates, Gail Model Risk Assessments, lung nodule screening criteria, triage protocols, and Press Ganey Scores. Patient navigation tools also include various types of tracking tools such as spreadsheets, task point, note templates, sticky notes, informal face sheets, chart reviews, Excel® spreadsheets, PowerPoint®, care coordination master schedules, Outlook® alerts, and various types of triage protocols.

Another type of navigation tool is a facility tool, which is any tool that is used within the facility for more than one patient and may include, for example, tools for measuring aspects of the navigation program itself, an example being a computer system, leadership meetings for program evaluation, process tools, pamphlets describing the navigator role with contact information, QA initiatives, and multidisciplinary meetings for consensus opinions regarding treatment planning. Professional standards were used as guidelines for metrics, and served as evaluative criteria for ongoing program development. Some of these included NAPC, CoC, NCCN, and ACOG. Computer tracking systems were utilized in most instances as a means of communication between the systems. Journey Forward (n.d.) was popular for use in survivorship. This is a free tool for oncology professionals to make tailored treatment plans. The CoC maintains that it meets the requirements identified by them and the IOM for important components of survivorship care (“Journey Forward,” n. d.). Human trackers included RN data specialists. Administrative tools for system analysis and goal formation included process maps, picture representation of program, specific navigation guidelines such as the NCOBC navigation steps. One program used an NCCP flowchart to standardize the navigation process amongst the different navigators within the system. Other programs included Practice Partner, NURSENAV, ARIUM, EQUICARE, EPIC, ASPEN, ACTS, CORDATTA, and BEACON. Patient navigation tools can be distinguished from facility tools in that patient tools are a tool used by the navigator, e.g., the NP, for gathering information about the particular patient under his or her care. Certain patient tools are published documents that can be used by the navigator to help triage the patient's needs. For example the navigator may use a tool to measure fatigue, a patient's performance status, the patient's personal risk of cancer, the patient's risk for high grade prostate cancer, etc. On the other hand a facility tool can be used for more than just a particular patient, but has a broader applicability within the facility, e.g., a computer software program such as some of those described herein. A facility tool can be used, for example, to communicate data amongst multiple people caring for the patient. For example in application of telemedicine technology can be used in the form of a system in which a patient physically remains in one place, while a provider is in another during treatment.

Yet another type of navigation tool is a “community tool,” which may include any one of a number of marketing materials, activities such as group meetings, and written materials such as community resource binders Certain community tools are tangible and physical, such as computer networks which interconnect individual patient computers or facility networks. Other community tools are human organizations, such as focus groups, which meet outside the facility and are part of the navigation process. For example, a focus group can be used by a navigator to collect information from patients in the community away from the particular facility, and the focus group is thus a community tool by which research is conducted. Certain focus groups have a set format that follows research methodology. When a nurse practitioner provides resources to a patient she may give the patient a research binder with a list of resources, talk to the patient on an individual basis to determine the patient's needs, then match particular resources with the patient's needs. The term “pulling resources” and “providing resources” covers more than just handing out information but also includes receiving information. For example if the patient were experiencing a crisis, had no family, and just received some upsetting medical news, e.g., a diagnosis of malignant tumor, the NP might call a social worker, and sit with the patient during the meeting with the social worker, call the MD and help the patient understand the diagnosis and options, spending more time with the patient than the MD would typically spend. If emergency services were needed such as emergency radiation, the NP would arrange for such services.

As discussed elsewhere herein, evaluation data can be transmitted by an individual using a computer. In the system 100 in FIG. 1 various client computers 101 are depicted, which can be, for example, a desktop or laptop or a tablet such as an iPad or a mobile phone that includes a computer such as an iPhone®. In certain specific embodiments herein, evaluation data can be entered into one of the client computers 101 by an individual. In certain embodiments described below, a patient uses one client computer 101 to enter his or her subjective evaluation data, and a navigator uses a different client computer 101 to enter his or her own subjective evaluation data. As exemplified in FIG. 2, a patient and navigator can each enter evaluation data using a client computer 101. Preferably, the evaluation data corresponds to certain specific health care aspects of the cancer that has been received by the patient, e.g., navigation “input.” For example, after barrier assessments are delivered to the patient by the navigator, the patient and navigator both can enter barrier evaluations, i.e., data evaluating those barrier assessments. Also, as discussed below, after triage is performed, the patient and navigator can both enter triage evaluations, i.e., data evaluating the triage provided.

Barrier Assessments:

“Barrier assessments” are defined herein as any barrier-related assessment, preferably barrier-focused, whether on an individual, facility, or community level. One type of barrier assessment is “a patient-level barrier assessment,” also referred to herein as a “patient barrier assessment,” which is an assessment of the barriers specific to a particular patient, such as lack of insurance or transportation. Another barrier assessment is “facility-level barrier assessment,” also referred to as an assessment or facility barriers or a “facility barrier assessment,” which is an assessment of the barriers specific to the facility that are also barriers to the patient. Assessment of facility barriers may include not only providing the patient with the identity and assessment of the particular resources within the particular facility, but also identifying ways to expedite and coordinate care within a particular facility where the patient is undergoing cancer care. Thus, assessment of facility barriers may in certain cases be proactive, and include not only identifying but also solving a particular barrier problem. For example, an NP may determine that a patient lacks personal transportation and so in order to overcome that barrier might provide the patient with information on public transportation which is a community resource. In certain cases, facility barrier assessments can be done using key connections amongst the facility staff. Such key connections may in some cases assist the NP in troubleshooting ways to overcome the facility barriers that impede patient care. One widely used means of implementing assessing patient needs to expedite facility care is through multidisciplinary team collaboration. For example, a navigator may interact with a neuro surgeon, radiation oncologist, medical oncologist, neuro radiologist and pathologist, along with a social worker and mid-level neuro surgery practitioners. Such interactions may involve sitting down and discussing patient cases, viewing images for the patients, reviewing brain imaging or spine imaging. The collaboration may also include identifying the best course of action to treat somebody's tumor whether it's malignant or benign using surgery, radiation or chemotherapy techniques. Individual patient, facility, and community barriers can be addressed with the key players that can help the patient obtain expedited care. Patient barriers to care can include things such as lack of transportation, insurance, social support, and housing. Facility barriers to care can include lack of resources within a particular facility such as lack of a radiation therapy department, insufficient staff personnel certified to give chemotherapy, treatment delays due to lack of staff to process the scheduling request in a timely manner, bottlenecks for processing important information such as pathology reports.

A third type of barrier assessment involves the assessment of community barriers, e.g., assessment of community resources outside the facility related to the patient. Certain community resources are related to patient resources. For example a patient may not have a car for transportation which is a patient barrier; and the community may not have an adequate bus system because the buses do not run after 6 pm when the patient finishes her chemotherapy treatment, which is a community barrier. The “community” is defined as any personnel or entities not employed by the medical facility. Community barrier assessment may thus include assessments of public education programs administered either within the hospital or in the community. Thus, a community barrier may be a service that is provided to patients within the walls of the facility but not controlled by the facility. For example, support groups and individual counseling for persons trying to quit smoking may be offered by non-hospital organizations, which qualify as community resources, regardless of whether they are administered within or away from the hospital.

After the navigator provides the patient with barrier assessment, either before or after the patient proceeds to another stage along the cancer care continuum, e.g., from the diagnosis stage to the treatment stage, the barrier assessment is evaluated, preferably by both the patient and the navigator, providing barrier assessment evaluation data. As noted above, the patient and navigator preferably each enter evaluation data using a client computer 101 as shown in FIG. 2. An illustrative list of barrier assessment evaluation propositions which can be answered by numerical evaluations, e.g., using a scale of 1-to-5, is set forth in TABLE 1 below.

TABLE 1
BARRIER ASSESSMENT EVALUATION DATA

| | |
|---|---|
| 1. | My psychosocial needs were met during my cancer care. |
| 2. | I have received a patient risk assessment from my navigator. |
| 3. | My insurance and funding were assessed to my satisfaction. |
| 4. | I received satisfactory treatment of my cancer diagnosis. |
| 5. | I was provided with adequate information on cancer staging. |
| 6. | I received adequate transportation to my treatment location. |
| 7. | My educational needs regarding my cancer care were met. |
| 8. | I was advised of my comorbidities (my medical conditions). |
| 9. | I received a global or comprehensive needs assessment with a focus on |
| | barriers to care. |
| 10. | I received a comprehensive patient assessment when I made initial contact |
| | with this facility or with my navigator. |
| 11. | I received a patient consultation. |
| 12. | I was fully advised about facility resources for receiving psychosocial care. |
| 13. | I received a facility cancer risk assessment. |
| 14. | I was provided with a summary of all resources provided by my cancer care |
| | facility for treating the cancer diagnosis. |
| 15. | I was provided with a summary of all resources provided by my cancer care |
| | facility for the cancer staging work-up. |
| 16. | I was provided with a summary of all resources provided by my cancer care |
| | facility transportation to my treatment location. |
| 17. | I was provided with a summary of all resources provided by my cancer care |
| | facility for education relative to my cancer care. |
| 18. | I was provided with a summary of all resources provided by my cancer care |
| | facility for resources addressing my comorbidities (medical conditions). |
| 19. | I was provided with a summary of comprehensive needs assessment with a |
| | focus on barriers to care provided by my cancer care facility. |
| 20. | I was provided with an adequate summary of resources |
| | available from my community for my psychosocial care |
| | before, during and after treatment. |
| 21. | I was provided with an adequate community risk assessment |
| | (for example, documentation). |
| 22. | I was provided with an adequate summary of resources |
| | available from my community for insurance and funding |
| | relative to my cancer care. |
| 23. | I was provided with an adequate summary of resources |
| | available from my community for my treatment of the cancer |
| | diagnosis. |
| 24. | I was provided with an adequate summary of resources |
| | available from my community for my cancer staging work-up. |
| 25. | I was provided with an adequate summary of resources |
| | available from my community for transportation to my |
| | treatment location. |
| 26. | I was provided with an adequate summary of resources |
| | available from my community for my education relative to my |
| | cancer care. |
| 27. | I was provided with an adequate summary of resources |
| | available from my community addressing my comorbidities |
| | (medical conditions). |
| 28. | I was provided with an adequate summary of global or |
| | comprehensive needs assessment provided by my community |
| | with a focus on barriers to care. |
| 29. | I was provided with a patient education binder with a list of |
| | cancer care resources. |
| 30. | I was adequately advised on how to communicate with the |
| | facility to update the patient care binder to incorporate |
| | additional patient care resources. |
| 31. | I was adequately advised on how to communicate with |
| | members of the community to update the patient care binder to |
| | incorporate additional patient care resources. |
| 32. | I was adequately advised regarding the need for the navigator |
| | to accompany me (for patient) to all appointments. |

Triage:

Triaging is an important aspect of navigation. Triage can be performed either during or after barrier assessment. Triage (triaging) is a term widely used by medical personnel that refers to a type of prioritizing of treatments, and that meaning is applied herein. Generally speaking, triaging involves determining which patient needs help most urgently. After triage is performed, the patient and navigator can both enter triage evaluations, i.e., provide data evaluating the triage provided. Generally speaking, there is a preferred sequence and order for cancer care due to the correlation between any diagnostic workup that is untimely, treatment initiation and disease progression. That is, for example, untimely care may result in undue disease progression, resulting in failure to achieve remission, cure and long term disease free survival. A timely triage process preferably enables design of a sequence of cancer care for processing a patient through a diagnostic work-up so that treatment can be initiated at an optimal time. Knowledge of the natural course of the disease guides the initial triage process, and the oncology nurse practitioner's (NP) prescriptive authority preferably expedites the process, which alleviates the need for physician order. Accordingly, there is an advantage in having an advanced practice RN (nurse practitioner or NP) navigator instead of an RN navigator. Because the NP or advanced practice RN navigator can write prescriptions for medications and testing, he/she is able to process the orders for care expeditiously. Alternatively the RN navigator has to call the physician to obtain orders for patient care. Thus the barriers to care should be addressed in the manner that is most logical for facilitating timely access. Accordingly, part of triage is factoring in the barriers that preferably were identified during the barrier assessment stage. Triaging can be applied relative to the patient, the facility, and the community.

Patient triaging can be performed either during or after the initial barrier assessment and can be performed at the same time as facility and community triaging. Triaging in general involves use of expert knowledge of factors that influence the patient's care, and also of the particular facility and community, e.g. connectivity to relevant contacts within the facility and community, to assist in overcoming barriers to care. A patient barrier frequently encountered during the navigation process is lack of time, particularly for navigators who have clientele with heavy navigational needs. To offset the barrier of lack of time, a navigator may utilize a triage process that identifies and gives priority to any patients who are at high risk for stagnating within the system and/or not completing their care due to unresolved navigation needs. An example is a patient who has problems obtaining insurance or funding for treatment may stagnate within the system. Accordingly, the desirability of removing an insurance or funding barrier for a particular stage in cancer care, e.g., diagnosis, may be a reason to triage that patient at the front of the line for diagnosis, assuming barriers remain for later treatment. Triaging in the context of navigation and as used herein preferably involves communicating the triage decisions to the patient, as well as the reasons for the decisions. For example, during triage, a navigator may inform the patient how his or her condition determines his or her order of treatment, as shown below in Table 2. By communicating the triage information, the navigator sets the patient's expectations, which is believed to result in a more satisfied patient than if the patient is not provided with such information. A navigator will, for example, communicate to the patient a timeframe on being visited by the medical oncologist before surgery, and the timeframe for when to expect a visit from the surgeon. If either timeframe is long, the patient may be advised that other patients with more urgent needs are being treated first, or some other basis for triaging.

Another type of triaging is facility triaging. Facility triaging is triaging that includes factors unrelated to the patient, e.g., involving other patients or features of the facility, e.g., medical equipment or hospital personnel. Thus, facility barriers identified in the preceding navigation stage (barrier assessment) may influence facility-level triage determinations. For example, where a facility only has limited use of particular equipment, or a MD only visits on a particular day, the patient's use of the equipment or meeting with the MD may be prioritized during the triage stage. Thus, a “first-come-first-served” system is more intuitive and fair from the perspective of the patient. However, after triage, once a patient is informed that another patient has received insurance approval for only diagnosis but not treatment, which requires the other patient to be treated first, the patient who is placed on a lower priority for diagnosis is expected to be more understanding. Thus, facility triaging takes into account the barriers faced not only by the patient in question but also the barriers faced by other patients within the facility.

A third type of triage is community triage, which is a type of triage influenced by aspects of the community of which the patient is a member. For example, a community defined as a high risk segment of the population might be endemic for lung cancer, have low literacy, or be a certain minority population. For example, it has been observed in at least one sampling of patients that less than 5% of outpatients who came in for screening mammography were Hispanic or Asian. Accordingly, in a heavily Hispanic or Asian community a patient qualifying as Hispanic or Asian might be triaged higher given the low screening within that community.

After the navigator performs the triage, and either before or after the patient proceeds to another stage along the cancer care continuum, the triage is evaluated, preferably by both the patient and the navigator. As discussed above, the patient and navigator each preferably enter triage evaluation data using a client computer 101 as shown in FIG. 2. An illustrative list of triage evaluation propositions is set forth in TABLE 2, each of which can be answered by numerical evaluations described above for the barrier assessment evaluations in TABLE 1.

TABLE 2
TRIAGE EVALUATION DATA

| | |
|---|---|
| 1. | My navigator adequately discussed with me the proper |
| | sequencing for meeting my psychosocial needs, insurance |
| | and funding, treatment for cancer diagnosis, cancer |
| | staging, and transportation to treatment in order of |
| | importance to ensure timely staging according to the |
| | resources within the facility. |
| 2. | My navigator adequately discussed with me the proper |
| | timeframe for meeting my psychosocial needs, insurance |
| | and funding, treatment of cancer diagnosis, cancer |
| | staging, and transportation to treatment in order of |
| | importance to ensure timely staging according to the |
| | resources within the facility. |
| 3. | My navigator adequately discussed with me the proper |
| | sequencing for meeting psychosocial needs, insurance |
| | and funding, treatment of cancer diagnosis, cancer |
| | staging, and transportation to treatment in order of |
| | importance to ensure timely staging according to the |
| | resources within the community. |
| 4. | My navigator adequately discussed with me the proper |
| | timeframe for meeting psychosocial needs, insurance |
| | and funding, treatment of cancer diagnosis, cancer |
| | staging, and transportation to treatment in order of |
| | importance to ensure timely staging according to |
| | resources within the community. |

Providing Resources:

After barrier assessment and triaging, the next stage of the navigation process described herein is providing resources to the patient, i.e., “resourcing.” As used herein “providing resources” (resourcing) includes pulling in resources for the patient, which may include both gathering information from the patient and providing information to the patient about the resources, including identifying or describing the resources or how to find or obtain them. The term “providing” as used herein means supplying, transmitting, or identifying, and also includes gathering or receiving. Whereas triaging involves identifying patient needs in order of importance, resourcing is a post-triage activity that involves matching patient needs with appropriate resources (relevant and useful information or identification of people who are helpful at a particular stage in the care process). The resourcing may be personal, or it may be from the facility or the community. Resources may be people who are able to assist the patient to obtain the resources that facilitate the completion of some phase of care, e.g., diagnostic process, in a timely manner. Resourcing may involve having frequent ongoing contact with the patient to determine the patient's ability to carry out their role in the process. Resourcing may also involve direct intervention by the navigator to troubleshoot and iron out any difficulties that the patient may have in obtaining the necessary resources in order of importance and in a timely fashion.

Providing resources may include care co-ordination, which is a central process by which navigators sought resources for the patient. Navigators can facilitate care-coordination among departments and specialists, appointment setters, family systems, research teams, insurance companies, state health departments, community resources for transportation, care providers in other states and any other resources that would be helpful to the patient. The navigator can both guide the patient to such resources and then also facilitate the use of the resources during the overall process to avoid treatment delays. Facility and community resources can be intertwined, and therefore can be addressed synergistically. For example, ineffective collection of appropriate resources for patients can result in treatment delays at both the facility and community level.

Providing resources in the context of the patient (“patient resourcing”) may include identifying information needed for that specific patient. In some cases, the patient resources relate to a particular stage in cancer care, e.g., diagnosis. Thus, in order to provide resources a navigator can determine if the patient has information needed for the particular stage in their cancer care, e.g., diagnosis. Specifically, for example, a navigator may discuss what they've been told by a particular physician and review any “path report” they might have received. Patient resourcing may include helping the patient make an appointment with one of the doctors, e.g., the surgeon. Patient resourcing may include providing the patient with a collection of documents containing information about breast cancer, the hospital, community resources for patients with breast cancer.

Resourcing may also include facility resourcing, a specific type of resourcing that involves pulling in resources specific to the facility, e.g., requiring interfacing with anyone in the medical facility who is involved in the care of the patient in any level of patient care. The navigator may need to communicate with many different levels of personnel in order to expedite and coordinate the patient care. For example, the navigator may need to gather resources from medical sales representatives, other nurse practitioners, primary care physicians, pulmonologists, medical oncology personnel, radiation oncology personnel, nurses, social workers, dieticians, the coordination between inpatient and outpatient. In the case of lung cancer patients who receive chemotherapy radiation at the same time as concomitant therapy, there is a need to coordinate, to make sure the patient has information about when his chemotherapy is set up to be started.

Yet another type of resourcing is community resourcing, i.e., providing resources from the community, which is any source outside the facility, e.g., an organization unaffiliated with the hospital in which the cancer care treatment is taking place, or not controlled by that hospital. Examples of community resourcing are identifying for the patient the resources in the community relevant to that particular patient's care program. For example, a navigator may provide the patient with a women's' service line, or an oncology service line. Community resourcing may include advising the patient about a local community educational presentation or a community support group.

After or during the resourcing, the patient and navigator can provide evaluations of the resourcing preferably using their client computers 101, in the same way barrier assessment evaluations and triage evaluations are provided, e.g., transmitted. An illustrative list of resourcing evaluation propositions is set forth in TABLE 3 any of which can be answered by the same numerical evaluations discussed above for the barrier assessment evaluations.

TABLE 3
RESOURCING EVALUATION DATA

| | |
|---|---|
| 1. | I was provided with key contacts to help meet my identified needs. |
| 2. | I was provided with key facility contacts to help iron out any problems within |
| | the facility to meet my needs. |
| 3. | I was provided with key community contacts to help meet my needs in the |
| | community. |
| 4. | Relationships were fostered between me and key contacts to help meet my |
| | identified needs. |
| 5. | Relationships were fostered between me and key facility contacts to help iron |
| | out problems within the facility to meet my needs. |
| 6. | Relationships were fostered between me and key community contacts to help |
| | meet my needs in the community. |
| 7. | The Nurse Practitioner's supervisor was actively involved in advocating for |
| | my personal needs as identified by my Nurse Practitioner. |
| 8. | The Nurse Practitioner's supervisor was actively involved in advocating for |
| | needs in the facility as identified by my Nurse Practitioner. |
| 9. | The Nurse Practitioner's supervisor was actively involved in advocating for |
| | my needs within the community as identified by my Nurse Practitioner. |
| 10. | The Nurse Practitioner “key physician collaborator” was actively involved in |
| | advocating for my personal needs as identified by my Nurse Practitioner. |
| 11. | The Nurse Practitioner “key physician collaborator” was actively involved in |
| | advocating for my needs within the community as identified by my Nurse |
| | Practitioner. |
| 12. | The Nurse Practitioner addressed my personal needs via multidisciplinary |
| | conference. |
| 13. | The Nurse Practitioner addressed contacts within the facility for meeting my |
| | needs via multidisciplinary conference. |
| 14. | The Nurse Practitioner addressed contacts within the community for meeting |
| | my needs via multidisciplinary conference. |
| 15. | The Nurse Practitioner communicated with the patient regularly to determine |
| | or review appropriateness of patient appointment schedules. |
| 16. | The Nurse Practitioner communicated with representatives of the facility |
| | regularly to determine or review appropriateness of patient appointment |
| | schedules. |
| 17. | The Nurse Practitioner communicated with representatives of the community |
| | regularly to determine or review appropriateness of patient appointment |
| | schedules. |
| 18. | The Nurse Practitioner communicated with the patient regularly to review |
| | timeliness of appointment schedules. |
| 19. | The Nurse Practitioner communicated with representatives of the facility |
| | regularly to determine timeliness of appointment schedules. |
| 20. | The Nurse Practitioner communicated with representatives of the community |
| | to determine timeliness of appointment schedules. |
| 21. | The Nurse Practitioner engaged in direct intervention when appropriate to |
| | iron out patient-related factors impeding access to appointments. |
| 22. | The Nurse Practitioner engaged in direct intervention when appropriate to |
| | iron out facility-related factors impeding access to appointments. |
| 23. | The Nurse Practitioner engaged in direct intervention when appropriate to |
| | iron out community-related factors impeding access to appointments. |
| 24. | The patient's overall plan of care was adjusted in response to any patient |
| | assessment changes. |
| 25. | The patient's overall plan of care relative to the facility was adjusted in |
| | response to any patient assessment changes. |
| 26. | The patient's overall plan of care relative to the community was adjusted in |
| | response to any patient assessment changes. |
| 27. | The Nurse Practitioner identified any needs for handing-off the patient to |
| | other medical personnel if applicable for patient follow-up along the cancer |
| | continuum from diagnosis to treatment. |
| 28. | The Nurse Practitioner identified any needs for handing-off the patient to |
| | other personnel within the facility if applicable for patient follow-up along the |
| | cancer continuum from diagnosis to treatment. |
| 29. | The Nurse Practitioner identified any needs for handing-off the patient to |
| | other personnel within the community if applicable for patient follow-up |
| | along the cancer continuum from diagnosis to treatment. |
| 30. | The Nurse Practitioner identified any needs for handing-off the patient to |
| | other personnel within the facility if applicable for patient follow-up along the |
| | cancer continuum from treatment to survivorship. |
| 31. | The Nurse Practitioner identified any needs for handing-off the patient to |
| | other personnel within the community if applicable for patient follow-up |
| | along the cancer continuum from treatment to survivorship. |
| 32. | The Nurse Practitioner assisted the patient to connect within the proper |
| | sequencing and timeframe; the resources for meeting the patient's |
| | psychosocial needs, insurance and funding, treatment of cancer, diagnosis, |
| | cancer staging, and transportation to treatment needs in order of importance to |
| | ensure timely diagnosis and staging. |
| 33. | The Nurse Practitioner assisted the patient to connect within the proper |
| | sequencing and timeframe; the resources for meeting the patient's |
| | psychosocial needs, insurance and funding, treatment of cancer, diagnosis, |
| | cancer staging, and transportation to treatment needs in order of importance to |
| | ensure timely staging according to resources within the facility. |
| 34. | The Nurse Practitioner assisted the patient to connect within the proper |
| | sequencing and timeframe; the resources for meeting the patient's |
| | psychosocial needs, insurance and funding, treatment of cancer, diagnosis, |
| | cancer staging, and transportation to treatment needs in order of importance to |
| | ensure timely staging according to resources within the community. |

Another aspect of resourcing involves resources pertinent to supportive care, which includes ongoing navigator support in the form of education/counseling, support group referrals, talking with the patient about confidential advice, empowering the patients to handle their affairs, and managing patient and/or navigator stress. As with the evaluations following barrier assessments, triaging and general resourcing, either during or after supportive care resourcing, the patient and navigator can provide evaluations of the supportive care resourcing preferably using their client computers 101. An illustrative list of supportive care resourcing evaluation propositions is set forth in TABLE 4 any of which can be answered by numerical evaluations as described above for barrier assessment evaluations listed in TABLE 1.

TABLE 4
SUPPORTIVE CARE RESOURCING EVALUATION DATA

| | |
|---|---|
| 1. | The patient was provided with adequate patient education/counseling while |
| | patient is undergoing the cancer diagnosis. |
| 2. | The patient was provided with adequate facility education/counseling |
| | regarding the patient plan of care while the patient is undergoing cancer |
| | diagnosis. |
| 3. | The patient was provided with adequate education to the community |
| | education/counseling regarding the patient plan of care during cancer |
| | diagnosis. |
| 4. | The patient was provided with adequate patient education/counseling while |
| | patient is undergoing the cancer treatment. |
| 5 | The patient was provided with adequate facility education/counseling |
| | regarding the patient plan of care while the patient was undergoing cancer |
| | treatment. |
| 6. | The patient was provided with adequate education to the community |
| | education/counseling regarding the patient plan of care while the patient was |
| | undergoing cancer treatment. |
| 7. | The patient was provided with adequate patient education/counseling while |
| | patient was undergoing the cancer survivorship care. |
| 8. | The patient was provided with adequate facility education/counseling |
| | regarding the patient plan of care while the patient was undergoing cancer |
| | survivorship care. |
| 9. | The patient was provided with adequate information about community |
| | education/counseling regarding the patient plan of care while the patient was |
| | undergoing cancer survivorship care. |
| 10. | The patient was provided with the identities of patient support groups for the |
| | patient during diagnosis. |
| 11. | The patient was provided with the identities of facility support groups for |
| | patients undergoing cancer diagnosis. |
| 12. | The patient was provided with the identities of community support groups for |
| | patients undergoing cancer diagnosis. |
| 13. | The patient was provided with the identities of patient support groups for the |
| | patient during cancer treatment. |
| 14. | The patient was provided with the identities of facility support groups for |
| | patients undergoing cancer treatment. |
| 15. | The patient was provided with the identities of community support groups for |
| | patients undergoing cancer treatment. |
| 16. | The patient was provided with the identities of patient support groups for the |
| | patient during cancer survivorship. |
| 17. | The patient was provided with the identities of facility support groups for |
| | patients undergoing cancer survivorship. |
| 18. | The patient was provided with the identities of community support groups for |
| | patients undergoing cancer survivorship. |
| 19. | The Nurse Practitioner served as a patient confidante regarding sensitive |
| | patient care topics during cancer diagnosis. |
| 20. | The Nurse Practitioner served as a communicator of sensitive patient care |
| | topics to facility providers with patient approval during cancer diagnosis. |
| 21. | The Nurse Practitioner helped the patient, to manage their stress. |
| 22. | The Nurse Practitioner advised the patient regarding measures to manage self- |
| | stress. |
| 23. | The Nurse Practitioner served as a communicator of sensitive patient care |
| | topics to community providers with patient approval during cancer diagnosis |
| | during cancer diagnosis. |
| 24. | The Nurse Practitioner served as a patient confidante regarding sensitive |
| | patient care topics during cancer treatment. |
| 25. | The Nurse Practitioner served as a communicator of sensitive patient care |
| | topics to facility providers with patient approval during cancer treatment. |
| 26. | The Nurse Practitioner served as a communicator of sensitive patient care |
| | topics to community providers with patient approval during cancer treatment. |
| 27. | The Nurse Practitioner served as a patient confidante regarding sensitive |
| | patient care topics during cancer survivorship. |
| 28. | The Nurse Practitioner served as a communicator of sensitive patient care |
| | topics to facility providers with patient approval during cancer survivorship. |
| 29. | The Nurse Practitioner served as a communicator of sensitive patient care |
| | topics to community providers with patient approval during cancer |
| | survivorship. |
| 30. | The Nurse Practitioner empowered the patient to handle his or her affairs, |
| | managing patient and/or navigator stress. |

Guidance:

The next stage within the navigation process is referred to as “guidance.” During this guidance stage, the navigator guides the patient to the next step in the cancer care process. After assessing barriers, triaging needs, and pulling in resources, the navigator provides guidance regarding the next step in his or her care. Broadly speaking this category speaks to the availability of the NP as an ongoing resource for guiding and directing the patient in all phases of the cancer journey. Additionally the navigator guides the patient to each phase of the cancer continuum and incorporates the assessment, triage, and pulling in resource steps to navigation during which she serves as an ongoing guide to facilitate and expedite the process. When the diagnostic phase is completed she guides the patient to the next step, which is treatment, then to survivorship.

The process of barrier-focused assessment, triaging needs, and pulling in resources can be ongoing in that one or more navigators may repeat the process along the cancer continuum e.g., from diagnosis to survivorship; contact with the patient took place from diagnosis to death. For other navigators, there may be contact in a specific phase of the cancer continuum such as the diagnostic or survivorship phase followed by handing the patient off to a provider who would see the patient through to the next step.

One aspect of guidance is guiding to the next step within a patient context, i.e., “patient guidance.” For example, in the case of a patient who is on the verge of having a biopsy taken, a navigator discusses the biopsy procedure with the patient and also describes the post-biopsy procedure, which may also be regarded as a follow-up, along with a physical examination. Thus, patient guidance in the form of guiding the particular patient to the next step includes describing the next step in the cancer care process without regard to the facility or community.

Facility guidance is a term that refers to guiding the patient to the next step in the cancer care continuum within the facility context, e.g., guiding the patient to a particular place within the facility or to a particular provider (e.g., an MD or midlevel provider (nurse practitioner or a physician's assistant) in order to facilitate cancer care both within and between all phases of the cancer continuum, for the purpose of expediting care. Facility guidance is particularly relevant during the post-treatment phase, after the patient has been treated, e.g., finished receiving radiation or chemotherapy treatment. As an example, during post-treatment placing the patient for survivorship care is important. Survivorship care requires the patient to take steps within the facility, to interact with other parts of the facility, e.g., the hospital. Survivorship care is often provided by a clinic operated by a nurse practitioner, either the patient's navigator or someone else, and/or may also include participating in a survivorship group program operated by the facility, and/or may also include receiving physician services either in primary care or oncology within the facility, other than the physician who either diagnosed or treated the patient. Thus, guidance to the next step in the facility context may include describing the various survivorship programs and explaining how to enlist in such programs, as well as recommending various physicians specializing in survivorship. A survivorship care plan is an important part of communicating patient treatment and follow-up care with the accepting survivorship provider. For example, certain physicians administer primary care oncology, in which the physician observes patients to see if they still have symptoms related to their diagnosis or treatment of their cancer. Alternatively, the navigator may inform the patient that a particular physician does osteoporosis management for patients who are on aromatase inhibitors (AI's) that require certain injections. The guidance within the facility during the survivorship stage may include providing the patient with a summary of her care, describing the types of active surveillance the patient can do, and recommending the patient provide the patient's primary care physician with the survivorship plan, and optionally preparing a letter directed to the patient's primary care physician describing the type of care required during survivorship, being alert to various symptoms, etc.

Also, guidance may include community guidance, i.e., guiding the patient to the next step within the context of the community, i.e., any actions that need to be taken outside the facility, or with an entity not affiliated with or controlled by the treatment facility, e.g., the hospital in which the patient was treated. For community guidance, the navigator lines up others who are in a position to provide the patient with resources within the community to facilitate and expedite patient care. Whereas providing resources as described above is performed by the navigator, the resources in the guidance step are provided by someone else, e.g., another individual or entity within the facility (facility guidance) or within the community (community guidance). As an example, guidance for next steps within the community includes arranging transportation, identifying times and places for support meetings that might take place in another part of the city or town where the facility is located. Sometimes guidance for next steps within the community includes coordinating the patient to go to another facility, e.g., the office of a surgeon who is not part of the treatment facility, and/or navigators in other facilities within the community.

After or while the navigator performs guidance to the next steps, the quality of the guidance can be evaluated, preferably by both the patient and the navigator, using a client computer 101 as shown in FIG. 1. An illustrative list of guidance evaluation propositions is set forth in TABLE 5, each of which can be answered by numerical evaluations as described above for barrier assessment evaluations listed in TABLE 1. Note that the propositions or statements in TABLE 5 refer to the patient in the third person whereas the propositions in TABLES 1-4 refer to the patient in the first person. The patient can be referred to either in the first person (“I”) or third person (“the patient”), at the option of the programmer or facility, and at least one specific embodiment includes both alternatives programmed in the software which can be changed by the facility administrator by selecting either “first person” or “third person.” In one or more other specific embodiments, the patient reads propositions that refer to the patient in the first person (“I”) and inputs the answers to the questions (e.g., using a 1-5 numerical answers) but the output, including any reports available to anyone reading the results, e.g., a hospital administrator or individual analyzing the evaluation data, is expressed in the third person (“the patient”).

TABLE 5
GUIDANCE EVALUATION DATA

| | |
|---|---|
| 1. | The Nurse Practitioner served as an ongoing guide for the patient in order to |
| | facilitate and expedite care within the context of the patient's needs in the |
| | diagnostic phase of cancer care. |
| 2. | The Nurse Practitioner served as an ongoing guide for the patient, in order to |
| | facilitate and expedite care for the patient within the context of the facility in |
| | the diagnostic phase of cancer care. |
| 3. | The Nurse Practitioner served as an ongoing guide for the patient in order to |
| | facilitate and expedite care for the patient within the context of the |
| | community in the diagnostic phase of cancer care. |
| 4. | The Nurse Practitioner served as an ongoing guide for the patient in order to |
| | facilitate and expedite care within the context of the patient's needs in the |
| | treatment phase of cancer care. |
| 5. | The Nurse Practitioner served as an ongoing guide for the patient in order to |
| | facilitate and expedite care for the patient within the context of the facility in |
| | the treatment phase of cancer care. |
| 6. | The Nurse Practitioner served as an ongoing guide for the patient in order to |
| | facilitate and expedite care for the patient within the context of the |
| | community in the treatment phase of cancer care. |
| 7. | The Nurse Practitioner served as an ongoing guide for the patient in order to |
| | facilitate and expedite care within the context of the patient's needs in the |
| | survivorship phase of cancer care. |
| 8. | The Nurse Practitioner served as an ongoing guide for the patient in order to |
| | facilitate and expedite care for the patient within the context of the facility in |
| | the survivorship phase of cancer care. |
| 9. | The Nurse Practitioner served as an ongoing guide for the patient in order to |
| | facilitate and expedite care for the patient within the context of the |
| | community in the survivorship phase of cancer care. |

As noted elsewhere herein, one important goal of the methods, systems, and CRM described herein is to improve the process by which patients move through the cancer care continuum, as opposed to merely improving diagnostic or treatment methods, techniques, or protocols, which is primarily a medical function. In one sense, it has been observed by the inventor that whereas individual performances of medical personnel may be excellent, those performances may be blunted by problems within the overall cancer care program. If the overall cancer care received by a patient is viewed as a chain, then even one weak link may have a dramatic negative impact on the care received by the patient. Tracking is a measurement of the navigation itself, including preferably the progress or outcomes of patient navigation. Such outcomes can be measured by metrics, which can be tracked using a variety of navigation tools.

Assessing Evaluation Data:

As noted elsewhere herein, at least certain embodiments of the methods, systems and CRM described herein includes assessing at least some of the evaluation data corresponding to one or more evaluated health care aspects of a particular patient. One way to assess such evaluation data is to formulate “metrics,” defined herein as a measurement of any aspect of the patient's cancer care that can be quantified and compared to something else. For example, as discussed elsewhere herein, certain timeliness factors such as diagnostic delays can be objectively measured in days, hours, and/or minutes and then compared to a benchmark, e.g., delays for other patients within the same facility or other facilities. Metrics such as timeliness factors expressed as days, hours, and/or minutes are typically objective and thus easily measurable. However, even certain subjective factors can be measured and thus be a part of the metrics discussed herein. For example, a patient may enter his or her subjective evaluation of how barrier assessments were conducted, in binary terms (good or bad) or on a scale of 1 to 5; accordingly the numbers become the metrics that can then be used for comparisons. A major goal for the navigation process is “high outcomes,” and metrics can be used to facilitate such high outcomes. Navigation tools can be used to facilitate tracking of these metrics. Preferably, however, “metrics” excludes any subjective answers to propositions such as evaluation data that includes a selection of numbers 1 through 5 in response to subjective propositions such as those listed in TABLES 1-5.

Metrics can be either patient metrics, or “system” metrics which included both the hospital system (i.e., the “facility”) and the community (everything outside the facility or unaffiliated with the facility). Tracking and metrics can be utilized throughout any phase of the navigation process in any stage of the cancer continuum.

“Patient metrics” are defined as metrics associated with the patient herself, rather than to other patients or to the facility or community, such as, for example, distress ratings, patient satisfaction scores, risk scores, referrals, lost to follow-up rates (where a patient simply does not get to an appointment, resulting in the navigator losing knowledge of where the patient is) treatment decisions, pathology report notifications, out migrations (when the patient decides to leave the treating facility and go elsewhere, e.g., to another hospital), and insurance authorizations. Patient satisfaction is a major goal in navigation. To the extent metrics includes subjective evaluations of the patient or navigator, the Press Ganey system may be employed as a non-exclusive means for measuring patient satisfaction, using “patient satisfaction scores.” For example, distress assessments (e.g., ratings) can be done for patients that meet criteria for having an assessment after screening, which may also include adjusting any issues causing the distress.

“Facility metrics” may also be measured, and those metrics relate to some aspect of the facility such as, for example, diagnostic metrics which may include measuring timely care such as the time for reporting pathology results to patient and/or provider, ordering staging tests in a timely manner, and obtaining and providing treatment consults. Thus, timing issues, such as delays between events along the cancer care continuum can be included as one of the facility metrics. A facility metric may be a combination or average of individual patient metrics in a particular facility. Unnecessary delays during the diagnostic phase may include a scenario where there is no need to obtain physician referral orders in view of nurse practitioner prescribing privileges that expedite the diagnostic work-up process. Other facility metrics may include the percentage(s) of patients lost to follow-up, STAR rehabilitation program referrals, number of patients seen, point along the cancer continuum, number of procedures and/or referrals, QA indefinable indicators such as sentinel node biopsies and DCIS, timely initiation of appointments, consistency of practice, face to face visits, phone calls, resource referrals, how long the case is open, admissions, discharges, number and types of interactions.

Another type of metrics is “community metrics,” which are similar to facility metrics but are objective measurements of some aspect of the community, e.g., the number of patients within a particular community that have participated in survivorship support groups.

In summary the major goal for the navigation process was to expedite patient care, and one way to measure the timeliness of the care delivered is through the use of metrics. Navigation tools can be tied closely with these metrics and facilitate the tracking of the metrics. Tracking and metrics can be used in all phases of the navigation process of assessing, triaging, needs, pulling in resources, and guiding to the next step. Utilization of metrics can expedite patient passage through the cancer continuum. Tracking and tracking tools can be used to facilitate keeping the navigator connected to the patient and system.

Examples of some useful navigation metrics are set forth in TABLE 6 below.

TABLE 6
NAVIGATION METRICS

| | |
|---|---|
| 1. | Distress Rating |
| 2. | Patient Satisfaction |
| 3. | Cancer Risk Evaluation |
| 4. | Number of New Referrals |
| 5. | Lost to Follow-up Rates |
| 6. | Treatment Decision Referrals In-house |
| 7. | Treatment Decision Referrals Our-sourced to Community |
| 8. | Pathology Report Notification to Patient |
| 9. | Pathology Report Notification to Provider |
| 10. | Outward Migration |
| 11. | Insurance Authorization |
| 12. | Quality of Care |
| 13. | Press Ganey Scores |
| 14. | Building Programs Around Expert Consensus Guidelines |
| 15. | Ordering Staging Work-in in a Timely Manner |
| 16. | Multidisciplinary Evaluation |
| 17. | Obtaining and Providing Treatment Consensus |
| 18. | Number of Face to Face Visits |
| 19. | Length of Time the Case is Open |
| 20. | Number of Admissions |
| 21. | Number of Discharges |
| 22. | Number of Phone Calls |
| 23. | Number of STAR or Other Rehabilitative Services |
| 24. | Number of Patients Seen at Each Point Along Cancer Continuum |
| 25. | Point of Cancer Continuum Patient is Currently at |
| 26. | Communicates a well-defined hands off process diagnosis to treatment if |
| | applicable to patient |
| 27. | Communicates a well-defined hands off process diagnosis to treatment if |
| | applicable to patient to facility |
| 28. | Communicates a well-defined hands off process diagnosis to treatment if |
| | applicable to patient to the community provider |
| 29. | Communicates a well-defined hands off process for treatment to survivorship |
| | if applicable to patient |
| 30. | Communicates a well-defined hands off process for treatment to survivorship |
| | if applicable to the facility provider |
| 31. | Communicates a well-defined hands off process for treatment to survivorship |
| | if applicable to the community provider |
| 32. | Patients Enrolled in a Research Protocol |
| 33. | Number of Procedure Referrals |
| 34. | Timely Initiation of Appointments |
| 35. | Consistency of Practice |
| 36. | Number and Type of Interactions |
| 37. | Compares Facility and Regional Statistics to State and National Statistics |
| 38. | Standardizes Measurement Tools for Data Mining and Managing Outcomes |
| 39. | Performs Patient Risk Assessments |
| 40. | Develops Chart Templates for Organization and Tracking Data |
| 41. | Institutes Standards of Care Such as Screening Tools for Organization, |
| | Tracking and/or Triage Purposes |
| 42. | Utilizes Feedback from Patient Satisfaction Surveys |
| 43. | Utilizes Spreadsheets for Organization and Tracking |
| 44. | Utilizes PowerPoint Tools |
| 45. | Utilizes a Master Schedule for Care-coordination |
| 46. | Utilizes the Outlook Alert System |
| 47. | Utilizes a Patient Tracking Tool |
| 48. | Triage Protocol Forms |
| 49. | Navigation Specific Computer Program Software |
| 50. | Homegrown Tailored Computer Program |
| 51. | Leadership Meetings for Program Evaluation |
| 52. | Systems Process Mapping |
| 53. | Pamphlet Describing Navigator Role with Contact Information |
| 54. | Quality Assurance Initiatives |
| 55. | Multidisciplinary Meetings to Determine Consensus Opinions Regarding |
| | Patient Management |
| 56. | Centralized Facility Computer Software Program |
| 57. | Human Trackers such as RN's that Track Metrics |
| 58. | Pictorial Representation of Navigation Program |
| 59. | Utilizes Marketing Tools in Community to Advertise Hospital Program |
| 60. | Holds Focus Groups with Community to Determine Need for Hospital |
| | Outreach Programs |
| 61. | Community Networking to Incorporate New Services into Patient/Facility |
| | Program |
| 62. | Development of a Resource Binder that Lists Community Resources |

Program Improvement:

After the evaluation data and metrics are analyzed, the results of the analysis may then be used to develop improvements to the cancer care program, including the patient navigation system. Such improvements may include delegation of non-nursing duties away from the Nurse Practitioner to ancillary personnel. Thus, the role of the Nurse Practitioner is frequently updated with the goal being for the Nurse Practitioner to function to the full level of his/her licensure. Examples of process improvement plans on a patient level are set forth in TABLE 7.

TABLE 7
IMPROVEMENT PLANS

| | |
|---|---|
| 1. | Use evaluation data to formulate process improvement plans on a facility |
| | level. |
| 2. | Use evaluation data to formulate process improvement plans on a community |
| | level. |
| 3. | Use metrics to expedite care along the cancer continuum against program |
| | standards. |
| 4. | Use correlational tends between and within the components of the navigation |
| | process to develop process improvement measures on a patient level. |
| 5. | Use correlational trends between and within the components of the navigation |
| | process to develop process improvement measures on a facility level. |
| 6. | Us correlational trends between and within the components of the navigation |
| | process to develop process improvement measures on a on a community |
| | level. |
| 7. | Further define correlations between patient satisfaction on a patient level to |
| | improve patient metrics. |
| 8. | Further define correlations between patient satisfaction on a facility level to |
| | improve facility metrics. |
| 9. | Further define correlations between patient satisfaction on a community level |
| | to improve community metrics. |
| 10. | Further define correlations that relate to timely care, on a patient level, to |
| | improve patient metrics. |
| 11. | Further define correlations that relate to timely care, on a facility level, to |
| | improve facility metrics. |
| 12. | Further define correlations that relate to timely care, on a community level, to |
| | improve community metrics. |
| 13. | Further define correlations that relate to connectivity, on a patient level, to |
| | improve patient metrics. |
| 14. | Further define correlations that relate to connectivity, on a facility level, to |
| | improve facility metrics. |
| 15. | Further define correlations that relate to connectivity, on a community level, |
| | to improve community metrics. |
| 16. | Further define correlations that relate to the navigation process metrics, on a |
| | patient level, to improve a selected metric. |
| 17. | Further define correlations that relate to the navigation process metrics, on a |
| | facility level, to improve a selected metric. |
| 18. | Further define correlations that relate to the navigation process metrics, on a |
| | community level, to improve a selected metric. |

A subset of program development is adjusting the navigation path or plan of care at the patient, facility, and/or community levels. For example, not only may the patient plan of care be adjusted, but also the way the facility and community handle the patient plan of care may be adjusted. Examples of how the roles of the navigator (e.g., Nurse Practitioner) can be adjusted are set forth in TABLE 8.

TABLE 8
ADJUSTMENTS

| | |
|---|---|
| 1. | Adjust the NP role in patient care so that the NP can practice to the highest |
| | level of his/her licensure in respect to the care of an individual patient. |
| 2. | Adjust the NP role in patient care so that the NP can practice to the highest |
| | level of his/her licensure in respect to facility care. |
| 3. | Adjust the NP role in patient care so that the NP can practice to the highest |
| | level of his/her licensure in respect to community care. |
| 4. | Adjust the NP role in the care of an individual patient so that the NP can |
| | practice to the highest level of his/her licensure in respect to navigation. |
| 5. | Adjust the NP role in the facility so that the NP can practice to the highest |
| | level of his/her licensure in respect to navigation.. |
| 6. | Adjust the NP role in the community so that the NP can practice to the highest |
| | level of his/her licensure in respect to navigation.. |
| 7. | Differentiate the RN navigator duties from the NP navigator duties in patient |
| | care. |
| 8. | Differentiate the RN navigator duties from the NP navigator duties with |
| | respect to navigation within the facility. |
| 9. | Differentiate the RN navigator duties from the NP navigator duties with |
| | respect to navigation within the community. |
| 10. | Differentiate nursing navigator duties from other navigator duties in |
| | navigating patient care. |
| 11. | Differentiate nursing navigator duties from other navigator duties in |
| | navigation within the facility. |
| 12. | Differentiate nursing navigator duties from other navigator duties in |
| | navigation within the community. |
| 13. | Create selected pre-set appointment slots for patient care. |
| 14. | Create selected pre-set appointment slots for patients referred within the |
| | facility. |
| 15. | Create selected pre-set appointment slots for patients referred within the |
| | community. |
| 16. | Remove selected pre-set appointment slots for patient care. |
| 17. | Remove selected pre-set appointment slots for patients referred within the |
| | facility for patient care. |
| 18. | Remove selected pre-set appointment slots for patients referred within the |
| | community for patient care. |
| 19. | Add symptom management to selected patient care. |
| 20. | Add symptom management to selected patient care within the facility. |
| 21. | Add symptom management to selected patient care within the community. |
| 22. | Add referrals for symptom management to role for patient care. |
| 23. | Add rehabilitation of patient to role of patient care. |
| 24. | Add rehabilitation of patient to role of patient care within the facility for |
| | cancer rehabilitative care. |
| 25. | Add rehabilitation of patient to role of patient care within the community for |
| | cancer rehabilitative care. |
| 26. | Add screening of the cancer patient to role for patient care. |
| 27. | Add screening of the cancer patient to role for patient care within facility. |
| 28. | Add screening of the cancer patient to role for patient care within community. |
| 29. | Add referral to initial consultation. |
| 30. | Add referral to initial consultation within the facility. |
| 31. | Add referral to initial consultation within the community. |
| 32. | Remove of minimize difficulties in accessing care resources to meet the |
| | patient's personal care needs. |
| 33. | Remove of minimize difficulties in accessing care resources within the |
| | facility. |
| 34. | Remove of minimize difficulties in accessing care resources within the |
| | community. |
| 35. | Tailor a patient's treatment plan based on the patient's individual needs. |
| 36. | Adjust the initial treatment plan according to changes in the patient's needs. |
| 37. | Identify additional resources in the community to meet the patient needs. |
| 38. | Make changes to improve facility metrics to more closely match existing care |
| | standards. |
| 39. | Make changes to remove or minimize patient care navigator stressors for |
| | individual patients. |
| 40. | Make changes to remove or minimize patient care navigator stressors within |
| | the facility. |
| 41. | Make changes to remove or minimize patient care navigator stressors within |
| | the community. |
| 42. | Identify and implement coping mechanisms for relieving patient care |
| | navigator stressors. |
| 43. | Identify and implement coping mechanisms for relieving patient care |
| | navigator stressors in relation to the facility. |
| 44. | Identify and implement coping mechanisms for relieving patient care |
| | navigator stressors in relation to the community. |
| 45. | Adjust survivorship care needs to survivorship care plan of care. |
| 46. | Adjust communication of survivorship care needs to facility personnel. |
| 47. | Adjust communication of survivorship care needs to community personnel. |
| 48. | Adjust survivorship health promotion care needs to survivorship care plan of |
| | care. |
| 49. | Adjust communication of survivorship health promotion care needs to facility |
| | personnel. |
| 50. | Adjust communication of survivorship health promotion care needs to |
| | community personnel. |
| 51. | Adjust bills or invoices for patient services delivered by |
| | facility personnel, including improved explanation of services |
| | rendered. |
| 52. | Adjust bills for patient services delivered by community personnel, including |
| | improved explanation of services rendered. |
| 53. | Improve skill of patient care personnel required for job and adjust goals for |
| | improving knowledge base. |
| 54. | Improve patient care on a provider level based on collaboration with key |
| | physicians |
| 55. | Improve patient care on a facility level based on collaboration with key |
| | physicians. |
| 56. | Improve patient care on a community level based on collaboration with key |
| | physicians. |
| 57. | Add ONS NP standards of care into practice on a patient level. |
| 58. | Add ONS NP standards of care into practice on a facility level. |
| 59. | Add ONS NP standards of care into practice on a community level. |
| 60. | Add multidisciplinary conferences to patient care on a patient level. |
| 61. | Add multidisciplinary conferences to patient care on a facility level. |
| 62. | Add multidisciplinary conferences to patient care on a community level. |
| 63. | Add, adjust, or revise instructions to nurses in the facility. |
| 64. | Add, adjust, or revise instructions to nurses in the community. |
| 65. | Improve education of facility ancillary personnel. |
| 66. | Add involvement of mentors to NP students to learn NP navigator role. |
| 67. | Add involvement of mentors to NP navigators to learn NP navigator role. |
| 68. | Add nursing research. |
| 69. | Provide direct input in reference to overseeing structure of navigation along |
| | the cancer continuum in reference to patient care. |
| 70. | Provide direct input in reference to overseeing the structure of navigation |
| | along the cancer continuum in reference to care in the facility. |
| 71. | Provide direct input in reference to overseeing the structure of navigation |
| | along the cancer continuum in reference to cancer community care. |
| 72. | Improve supervision of office personnel within facility. |
| 73. | Meet with, and obtain live feedback from, selected patients with the cancer |
| | diagnosis that are being navigated. |
| 74. | Meet with, and obtain live feedback from, selected patients with a cancer |
| | diagnosis that are being navigated on consult basis only. |
| 75. | Set appointments for the patient within the facility. |
| 76. | Set appointments for the patient within the community. |
| 77. | Oversee patient appointments to ensure timely access. |
| 78. | Meet with patient prior to presenting to physician in the facility. |
| 79. | Meet with patient prior to physician in the community. |
| 80. | Adjust treatment options to the patient prior to the physician. |
| 81. | Meet with patient prior to initial cancer appointment. |
| 82. | Provide facility clinical trial information to the patient. |
| 83. | Provide community clinical trial information to the patient. |

Additionally, based on the navigation experience, evaluation data may include the patient's evaluation of the nurse practitioner, e.g., the Navigator. Examples of Nurse Practitioner evaluation data are set forth in TABLE 9, which can be answered with numerical evaluation answers, similar to the answers used for barrier assessment, triaging, resourcing, and guidance. TABLE 9 includes different categories of assessment.

TABLE 9
PATIENT EVALUATION OF NURSE PRACTITIONER (E.G., NAVIGATOR)
Category 1

| | |
|---|---|
| 1. | My nurse practitioner addressed my psychosocial needs. |
| 2. | My nurse practitioner did a family history and counseled me about my risk of |
| | cancer. |
| 3. | My nurse practitioner did a family history and counseled me about my |
| | family's risk of cancer. |
| 4. | My nurse practitioner directed me or provided me helpful resources to address |
| | my insurance and funding needs. |
| 5. | My nurse practitioner explained my treatment of my cancer diagnosis. |
| 6. | My nurse practitioner explained to my cancer staging. |
| 7 | My nurse practitioner arranged or provided me with helpful resources for my |
| | transportation to treatment. |
| 8. | My nurse practitioner addressed my educational needs. |
| 9. | My nurse practitioner talked to me about how my non cancer |
| | diagnosis/diagnoses would impact my treatment. |
| 10. | My nurse practitioner talked to my about any issues that influenced my cancer |
| | care. |
| 11. | My nurse practitioner reviewed my needs on my first contact |
| 12. | My nurse practitioner talked to me about facility resources for my |
| | psychosocial needs. |
| 13. | My nurse practitioner talked to me about facility resources that address my |
| | cancer risk. |
| 14. | My nurse practitioner talked to me or directed me to facility resources that |
| | will assist me with insurance and funding for my cancer care. |
| 15. | My nurse practitioner talked to me or provided direction to me about facility |
| | resources for treating my cancer. |
| 16. | My nurse practitioner talked to me about facility resources for the cancer |
| | staging work-up. |
| 17. | My nurse practitioner talked to me about facility resources for transportation |
| | to treatment. |
| 18. | My nurse practitioner talked to me about facility resources for education. |
| 19. | My nurse practitioner talked to me about facility resources that address my |
| | other non-cancer diagnosis/diagnoses. |
| 20. | My nurse practitioner talked to me about the all of my needs and matched me |
| | with people in the facility that could help me provide ways to overcome the |
| | obstacles that interfere with me getting cancer treatment. |
| 21. | My nurse practitioner provided me with community resources for my |
| | psychosocial care. |
| 22. | My nurse practitioner talked to me about cancer risk factors in the |
| | community. |
| 23. | My nurse practitioner talked to me about community resources for insurance |
| | and funding. |
| 24. | My nurse practitioner talked to me about community resources for my cancer |
| | staging work-up. |
| 25. | My nurse practitioner talked to me about community resources for |
| | transportation to treatment. |
| 26. | My nurse practitioner talked to me about community resources for education. |
| 27. | My nurse practitioner talked to me about community resources that address |
| | my other non-cancer diagnosis/diagnoses. |
| 28. | My nurse practitioner talked to me about the all of my needs and matched me |
| | with people in the community that could help me provide ways to overcome |
| | the obstacles that interfere with my getting cancer treatment. |
| 29. | My nurse practitioner presented a patient education binder showing a list of |
| | cancer care resources that meet my personal needs. |
| 30. | My nurse practitioner presented a patient education binder showing a list of |
| | cancer care resources in the facility that meets my needs. |
| 31. | My nurse practitioner presented a patient education binder showing a list of |
| | cancer care resources in the community that meet my needs. |
| 32. | My nurse practitioner addressed the need to accompany me to my |
| | Appointments. |

Category 2

| | |
|---|---|
| 33. | My nurse practitioner ranked my personal psychosocial, insurance and |
| | funding, treatment of cancer diagnosis, cancer staging, and transportation to |
| | treatment in order of importance to guide me to receive prompt care. |
| 34. | My nurse practitioner discussed the proper timeframe for meeting |
| | psychosocial, insurance and funding, treatment of cancer diagnosis, cancer |
| | staging, and transportation to treatment in order of importance to ensure |
| | timely staging. |

Category 3

| | |
|---|---|
| 35. | My nurse practitioner identified key contacts that helped me meet my |
| | identified needs. |
| 36. | My nurse practitioner identified key facility contacts that helped me iron out |
| | problems within the facility to meet my needs. |
| 37. | My nurse practitioner identify key community contacts that helped me meet |
| | my needs in the community. |
| 38. | My nurse practitioner had a good relationship with key contacts that will help |
| | meet my identified needs. |
| 39. | My nurse practitioner had a good relationship with key facility contacts that |
| | helped iron out problems within the facility to meet my needs. |
| 40. | My nurse practitioner had a good relationship with key community contacts |
| | that helped meet my needs in the community. |
| 41. | My NP worked with my physician who assisted the NP in helping me meet |
| | my identified needs. |
| 42. | My NP worked with my physician who assisted the NP in helping me meet |
| | my identified needs within the facility. |
| 43. | My NP worked with my physician who assisted the NP in helping me meet |
| | my identified needs within the community. |
| 44. | My NP addressed my needs in a multidisciplinary conference. |
| 45. | My NP communicated with me regularly to review the appropriateness of my |
| | patient appointment schedule. |
| 46. | My NP communicates with people within the facility regularly to determine |
| | appropriateness my appointment schedule. |
| 47. | My NP communicates with people within the community to determine |
| | appropriateness of my appointment schedule. |
| 48. | My NP communicated with me regularly to review timeliness of my |
| | appointment schedule. |
| 49. | My NP communicated with the facility regularly to ensure the timeliness of |
| | my appointment schedule. |
| 50. | My NP communicates with community resources regularly to determine |
| | timeliness my appointment schedule. |
| 51. | My NP directly intervened to iron out patient factors that got in the way of my |
| | accessing my appointments. |
| 52. | My NP directly intervened to iron out facility factors that got in the way of |
| | my access to appointments. |
| 53. | My NP directly intervened to iron out community factors that got in the way |
| | of my access to appointments. |
| 54. | My NP regularly readjusted my overall plan of care if my needs changed. |
| 55. | My NP readjusted my overall plan of care within the facility if my needs |
| | changed. |
| 56. | My NP readjusted my overall plan of care within the community if my needs |
| | changed. |
| 57. | My NP identified handoff if applicable to my follow-up treatment provider |
| | after my cancer was diagnosed. |
| 58. | NP identifies handoff if applicable to my follow-up treatment provider in the |
| | facility after my cancer was diagnosed. |
| 59. | NP identifies handoff if applicable to my follow-up treatment provider in the |
| | community after my cancer was diagnosed. |
| 60. | My NP identified handoff if applicable to my follow-up survivorship provider |
| | after I received my cancer treatment. |
| 61. | NP identified handoff if applicable to my follow-up survivorship provider in |
| | the facility after I received my cancer treatment. |
| 62. | My NP identified handoff if applicable to my follow-up survivorship provider |
| | in the community after I received my cancer treatment. |
| 63. | My NP assisted me to connect with within the proper sequencing and |
| | timeframe; the resources for meeting my psychosocial, insurance and funding, |
| | treatment of cancer, diagnosis, cancer staging, and transportation to treatment |
| | needs in order of importance. |
| 64. | My NP assisted me to connect with within the proper sequencing and |
| | timeframe; the resources for meeting my psychosocial, insurance and funding, |
| | treatment of cancer, diagnosis, cancer staging, and transportation to treatment |
| | needs in order of importance within the facility. |
| 65. | My NP assisted me to connect with within the proper sequencing and |
| | timeframe; the resources for meeting my psychosocial, insurance and funding, |
| | treatment of cancer, diagnosis, cancer staging, and transportation to treatment |
| | needs in order of importance within the community. |
| 66. | My NP ensured that I had a timely diagnosis and staging of my cancer. |
| 67. | My NP provided education/counseling while I was undergoing my cancer |
| | diagnosis. |
| 68. | My nurse practitioner provides facility education/counseling regarding my |
| | plan of care while I was undergoing my cancer diagnosis. |
| 69. | My nurse practitioner provides education to the community |
| | education/counseling regarding my cancer diagnosis. |
| 70. | My nurse practitioner provides patient education/counseling I was undergoing |
| | the cancer treatment. |
| 71. | My nurse practitioner provides facility education/counseling regarding my |
| | plan of care while I was undergoing cancer treatment. |
| 72. | My nurse practitioner provided education to the community |
| | education/counseling regarding my plan of care while I was undergoing |
| | cancer treatment. |
| 73. | My nurse practitioner provides patient education/counseling to me while I |
| | was undergoing the cancer survivorship care. |
| 74. | My nurse practitioner provided facility education/counseling regarding my |
| | plan of care while I was undergoing cancer survivorship care. |
| 75. | My nurse practitioner provided education to the community regarding the |
| | patient plan of care while I was undergoing cancer survivorship care. |
| 76. | My nurse practitioner identified patient support services for me during my |
| | cancer diagnosis. |
| 77. | My nurse practitioner provided information on facility support groups for me |
| | during my cancer diagnosis. |
| 78. | My nurse practitioner provided community support groups for me when I was |
| | undergoing my cancer diagnosis. |
| 79. | My nurse practitioner identified patient support groups for me during my |
| | cancer treatment. |
| 80. | My nurse practitioner provided information on facility support groups for |
| | patients while I was undergoing my cancer treatment. |
| 81. | My nurse practitioner provided information on community support groups for |
| | me while was undergoing cancer treatment. |
| 82. | My nurse practitioner identified patient support groups for me during cancer |
| | survivorship. |
| 83. | My nurse practitioner provided information on facility support groups for |
| | cancer survivorship. |
| 84. | My nurse practitioner provided information on community support groups for |
| | patient undergoing cancer survivorship. |
| 85. | My nurse practitioner served as a patient confidante regarding sensitive |
| | patient care topics during my cancer diagnosis. |
| 86. | My nurse practitioner served as a communicator of sensitive patient care |
| | topics to facility providers with patient approval during my cancer diagnosis. |
| 87. | My nurse practitioner served as a communicator of sensitive patient care |
| | topics to community providers with patient approval during my cancer |
| | diagnosis. |
| 88. | My nurse practitioner helped me manage my stress. |
| 89. | Serves as a patient confidante regarding sensitive patient care topics during |
| | cancer treatment. |
| 90. | Serves as a communicator of sensitive patient care topics to facility providers |
| | with patient approval during cancer treatment. |
| 91. | Serves as a communicator of sensitive patient care topics to community |
| | providers with patient approval during cancer treatment. |
| 92. | My nurse practitioner served as a patient confidante regarding sensitive |
| | patient care topics during cancer survivorship. |
| 93. | My nurse practitioner served as a communicator of sensitive patient care |
| | topics to facility providers with patient approval during cancer survivorship. |
| 94. | My nurse practitioner served as a communicator of sensitive patient care |
| | topics to community providers with patient approval during cancer |
| | survivorship. |
| 95. | My nurse practitioner empowered me to handle my affairs. |

Category 4

| | |
|---|---|
| 96. | My nurse practitioner was ongoing guide for me to facilitate and expedite |
| | care for to meet my personal needs in the diagnostic phase of my cancer care. |
| 97. | My nurse practitioner was ongoing guide for me to facilitate and expedite |
| | care for to meet my needs within the facility in the diagnostic phase of my |
| | cancer care. |
| 98. | My nurse practitioner was ongoing guide for me to facilitate and expedite |
| | care for me to meet my needs within the community in the diagnostic phase |
| | of my cancer care. |
| 99. | My nurse practitioner was ongoing guide for me to facilitate and expedite |
| | care for to meet my personal needs in the treatment phase of my cancer care. |
| 100. | My nurse practitioner was ongoing guide for me to facilitate and expedite |
| | care for to meet my needs within the facility in the treatment phase of my |
| | cancer care. |
| 101. | My nurse practitioner was ongoing guide for me to facilitate and expedite |
| | care for to meet my needs within the community in the treatment phase of my |
| | cancer care. |
| 102. | My nurse practitioner was ongoing guide for me to facilitate and expedite |
| | care for to meet my personal needs in the survivorship phase of my cancer |
| | care. |
| 103. | My nurse practitioner was ongoing guide for me to facilitate and expedite |
| | care for to meet my needs within the facility in the survivorship phase of my |
| | cancer care. |
| 104. | My nurse practitioner was ongoing guide for me to facilitate and expedite |
| | care for to meet my needs within the community in the survivorship phase of |
| | my cancer care. |

Category 5

| | |
|---|---|
| 105. | My nurse practitioner helped me cope with my distress experienced with my |
| | cancer diagnosis. |
| 106. | I was satisfied with the care received from my nurse practitioner. |
| 107. | My nurse practitioner reviewed with me my cancer risk Evaluation. |
| 108. | This is the first time that I have been seen in this facility for cancer. |
| 109. | I completed all of my cancer care at this institution. |
| 110. | I received my cancer treatment at this facility. |
| 111. | My cancer treatment was done at another facility. |
| 112. | My nurse practitioner notified me when she said she would of my pathology. |
| 113. | My PCP was knowledgeable of my pathology. |
| 114. | I received prompt notification of my insurance authorization by my NP. |
| 115. | I felt that I received a high level of care by my NP. |
| 116. | I was asked to rate my NP with a questionnaire. |
| 117. | I feel like that cancer care was in line with expert recommendations for my |
| | type of cancer. |
| 118. | I felt like my diagnostic tests were done in a timely manner. |
| 119. | My NP communicated with my cancer care team. |
| 120. | My NP was knowledgeable of my whole treatment plan. |
| 121. | My NP provided me with face to face visits as needed. |
| 122. | My NP was easily reachable by phone. |
| 123. | My NP addressed my |
| 124. | My NP saw me through diagnosis, treatment, and the survivorship phase of |
| | my cancer care. |
| 125. | My NP communicated to me my diagnostic care and the rationale for my |
| | treatment. |
| 126. | My NP communicated to the facility an in-depth review of my diagnostic care |
| | and the rationale for my treatment. |
| 127. | My NP communicated to the accepting community treatment provider an in- |
| | depth review of my diagnostic care and the rationale for my treatment. |
| 128. | My NP communicated to me my survivorship care and the rationale for my |
| | treatment. |
| 129. | My NP communicated to the facility an in-depth review of my survivorship |
| | care and the rationale for my treatment. |
| 130. | My NP communicated to the accepting community treatment provider an in- |
| | depth review of my survivorship care and the rationale for my treatment. |
| 131. | My nurse practitioner enrolled me in a research protocol. |
| 132. | My nurse practitioner referred me for my procedures. |
| 133. | My appointments were scheduled in a timely manner. |
| 134. | I could ask my nurse practitioner for help with anything related to my cancer |
| | diagnosis. |
| 135. | My nurse practitioner discussed with me how my survival rate compared to |
| | other patients in the facility, region, and nation. |
| 136. | My nurse practitioner performs a cancer risk assessment on me based on my |
| | level of illness that was communicated to me. |
| 137. | My nurse practitioner performs a cancer risk assessment on me based on my |
| | family history that was communicated to me. |
| 138. | My nurse practitioner performs a genetic risk assessment on me based on my |
| | family history that was communicated to me. |
| 139. | My nurse practitioner communicated with me on a regularly basis to track my |
| | progress with my cancer care. |
| 140. | My satisfaction with my cancer care was reviewed with a survey. |
| 141. | I received feedback from my satisfaction with my cancer care survey results. |
| 142. | My nurse practitioner seemed organized in that he/she was able to access my |
| | care without me having to wait too long. |
| 143. | My nurse practitioner always helped me co-ordinate my care. |
| 144. | My nurse practitioner tracked my progress during the diagnostic phase of my |
| | illness. |
| 145. | My nurse practitioner tracked my progress during the treatment phase of my |
| | illness. |
| 146. | My nurse practitioner tracked my progress during the survivorship phase of |
| | my illness. |
| 147. | My nurse practitioner tracked my progress during all phases of my cancer |
| | care. |
| 148. | My nurse practitioner provided me with written information that mapped out |
| | each stage of my cancer treatment. |
| 149. | My nurse practitioner provided me with written information that mapped out |
| | each stage of my cancer treatment with timeframes for completing each |
| | appointment. |
| 150. | My nurse practitioner provided me with written information that mapped out |
| | each stage of my cancer treatment with timeframes for completing each |
| | appointment; and this information was computer generated. |
| 151. | My nurse practitioner gave me a pamphlet describing his/her navigator role |
| | with contact information. |
| 152. | My nurse practitioner presented me to multidisciplinary meetings to |
| | determine the best way to handle my cancer care. |
| 153. | My nurse practitioner gave me feedback from multidisciplinary meetings to |
| | that were used to determine the best way to handle my cancer care. |
| 154. | My community practitioners were able to access my cancer care by computer. |
| 155. | My community practitioners were had all my up to date information on my |
| | cancer care. |
| 156. | I received a phone call from someone in the facility where I was treated for |
| | my cancer care, who notified me when my screening tests were due. |
| 157. | I received a pictorial representation of my oncology navigation program. |
| 158. | I heard about my nurse practitioner from marketing advertisement. |
| 159. | My nurse practitioner holds focus groups in the community to determine the |
| | need for new programs. |
| 160. | If a resources was not available to help me get through my cancer care my |
| | nurse practitioner contacted other support services in the community that |
| | would assist me. |
| 161. | My nurse practitioner gave me a resource binder that lists community |
| | resources. |
| 162. | I received regular updates on my resource binder that lists community |
| | resources. |

Category 6

| | |
|---|---|
| 163. | My nurse practitioner regularly reviewed with me my satisfaction with cancer |
| | care and adjusted my plan of care based on my current needs. |
| 164. | My nurse practitioner wrote prescriptions for me when needed. |
| 165. | My nurse practitioner gave me a clinic appointment to see his/her when my |
| | health needs required this. |
| 166. | My nurse practitioner did a performed a history and did a physical exam on |
| | me when the health care needs required this. |
| 167. | I was serviced by an RN navigator in addition to my NP navigator. |
| 168. | I understand the difference between an RN navigator and a Nurse practitioner |
| | navigator. |
| 169. | I was able to call and get an appointment with my nurse practitioner without a |
| | long wait time. |
| 170. | My nurse practitioner does not have pre-set appointment slots for patient care. |
| 171. | My nurse practitioner prescribed medication for me when I had cancer related |
| | symptoms. |
| 172. | My nurse practitioner helped me with my rehabilitation needs when after my |
| | treatment was finished. |
| 173. | I received a referral for my cancer rehabilitative care by my NP that was |
| | within the facility. |
| 174. | I received a referral for my cancer rehabilitative care by my NP that was |
| | within the community. |
| 175. | My nurse practitioner screened me for cancer during my initial visit. |
| 176. | My nurse practitioner screened me for cancer during my treatment. |
| 177. | My nurse practitioner screened me for cancer during survivorship. |
| 178. | My nurse practitioner saw me in the outpatient setting. |
| 179. | My nurse practitioner saw me in the in-patient setting. |
| 180. | I continued to see my nurse practitioner after my treatment ended by making |
| | an appointment. |
| 181. | My nurse practitioner ironed out difficulties in accessing care resources to |
| | meet my personal care needs related to my cancer diagnosis. |
| 182. | My nurse practitioner ironed out difficulties my accessing care resources |
| | within the facility. |
| 183. | My nurse practitioner ironed out difficulties in accessing care resources |
| | within the community. |
| 184. | My nurse practitioner tailoring the treatment plan based on my individual |
| | needs. |
| 185. | My nurse practitioner readjusting my treatment plan according to changes in |
| | my needs. |
| 186. | My nurse practitioner sought out resources in the community to meet my |
| | needs. |
| 187. | My nurse practitioner identified stressors in my life. |
| 188. | My nurse practitioner identified my patient care stressors, in relationship to |
| | the facility. |
| 189. | My nurse practitioner identified my patient care navigator stressors in |
| | relationship to the community. |
| 190. | My nurse practitioner identified coping measures for relieving for relieving |
| | my cancer related stressors. |
| 191. | My nurse practitioner identified coping measures for relieving my cancer |
| | patient care issues related to the facility. |
| 192. | My nurse practitioner identified coping mechanisms for relieving my cancer |
| | patient care issues related to community factors. |
| 193. | My nurse practitioner identified my personal survivorship care needs on |
| | survivorship care plan of care. |
| 194. | My nurse practitioner communicated my personal survivorship care needs to |
| | facility personnel. |
| 195. | My nurse practitioner communicated my personal survivorship care needs to |
| | community personnel. |
| 196. | My nurse practitioner identified my survivorship health promotion or |
| | wellness care needs on my survivorship care plan of care. |
| 197. | My nurse practitioner communicated my survivorship health promotion care |
| | needs to facility personnel |
| 198. | My nurse practitioner communicated my survivorship health promotion care |
| | needs to community personnel. |
| 199. | My nurse practitioner billed his/her services. |
| 200. | My nurse practitioner billed for his/her services that I used in the community. |
| 201. | My nurse practitioner answered my questions related to my cancer to my |
| | satisfaction. |
| 202. | My nurse practitioner collaborated with my key physician/physicians for |
| | ways to improve patient my care. |
| 203. | My nurse practitioner helped coordinate my care based on my needs that we |
| | discussed for my multidisciplinary conference. |
| 204. | My nurse practitioner helped coordinate my care based on my needs that we |
| | discussed at the multidisciplinary conference with members of the facility. |
| 205. | My nurse practitioner helped coordinate my care based on my needs that we |
| | discussed at the multidisciplinary conference with members of the |
| | community. |
| 206. | My nurse practitioner provides instruction for my care to nurses in the |
| | facility. |
| 207. | My nurse practitioner provided instruction for my care to nurses in the |
| | community. |
| 208. | My nurse practitioner educates facility non nurse personnel about my care. |
| 209. | My nurse practitioner researched answers to my questions that she/did not |
| | know that answer to. |
| 210. | My nurse practitioner directed the office personnel regarding my cancer care. |
| 211. | My nurse practitioner met with me at the initial stage of my cancer diagnosis. |
| 212. | My nurse practitioner navigator met with me the first time to address a certain |
| | problem related to my cancer care and thereafter on an as needed basis. |
| 213. | My nurse practitioner set appointments for me within the facility. |
| 214. | My nurse practitioner sets appointments for me within the community. |
| 215. | My nurse practitioner oversaw my patient appointments to be sure that I did |
| | not have long wait times. |
| 216. | My nurse practitioner saw me prior to presenting to the facility providers in |
| | the facility. |
| 217. | My nurse practitioner presented me prior to the physician for care in the |
| | community. |
| 218. | My nurse practitioner presented treatment options to me prior to the |
| | physician. |
| 219. | My nurse practitioner saw me initially on my first cancer appointment. |
| 220. | My nurse practitioner presented facility clinical trial information to me. |
| 221. | My nurse practitioner presented community clinical trial information to me. |
| | My nurse practitioner saw me through all of my cancer care from diagnosis to |
| | treatment to survivorship. |
| 223. | My nurse practitioner saw me through only one phase of my cancer care |
| | (cancer diagnosis). |
| 224. | My nurse practitioner saw me through only one phase of my cancer care |
| | (cancer treatment). |
| 225. | My nurse practitioner saw me through only one phase of my cancer care |
| | (cancer survivorship) |

1. A method in a computing system having one or more programmable processors communicatively coupled to memory and a database on a computer readable medium for adjusting a role of a cancer care navigator, comprising:
receiving over a computer network from an input device data representing a response to an evaluation request, the data representing the response comprising:
a navigation step identifier that uniquely identifies a navigation step of cancer care delivered by the cancer care navigator to g patient, wherein the navigation step is selected from the group consisting of one or more barrier assessments, triaging, resourcing, and guidance performed for the patient;
a proposition identifier that uniquely identifies a proposition; and
an evaluation value selected from a set of evaluation values and by a person with knowledge of the navigation step of cancer care;
storing in an evaluation response data structure in the database:
the navigation step identifier in an evaluation navigation step identifier field;
the proposition identifier in an evaluation proposition identifier field; and
the evaluation value in an evaluation value field;
retrieving data representing a stored response from data representing a set of one or more stored responses from the evaluation response data structure in the database, wherein the data representing the stored response comprises:
the navigation step identifier in the evaluation navigation step identifier field;
the proposition identifier in the evaluation proposition identifier field; and
the evaluation value in the evaluation value field;
retrieving data representing a set of one or more benchmark responses from the evaluation response data structure in the database, wherein the data representing each benchmark response comprises:
a benchmark proposition identifier in a benchmark proposition identifier field, where the benchmark proposition identifier and the proposition identifier of the stored response are equal; and
a benchmark evaluation value in a benchmark evaluation value field;
aggregating the benchmark evaluation values of the data representing the set of one or more benchmark responses into an aggregate benchmark evaluation value;
calculating an evaluation delta, wherein the evaluation delta is equal to the difference between the evaluation value of the stored response and the aggregate benchmark evaluation value;
retrieving data representing a proposition threshold from data representing a set of one or more proposition thresholds from a proposition threshold data structure in the database, wherein the data representing the proposition threshold comprises:
a proposition identifier in a proposition identifier field; and
a threshold in a threshold field;
assessing whether the evaluation delta exceeds the threshold
retrieving data representing an adjustment proposal from data representing a set of one or more adjustment proposals from an adjustment proposal data structure in the database, wherein the data representing the adjustment proposal comprises:
an adjustment proposal proposition identifier in an adjustment proposal procession identifier field, where the adjustment proposal proposition identifier and the threshold proposition identifier are equal; and
proposal text in a proposal field, the proposal text reciting one or more adjustments to the role of the cancer care navigator in delivering one or more navigation steps of cancer care; and
generating data representing a navigator role adjustment proposal, wherein data representing the navigator role adjustment proposal comprises:
one or more navigator identifiers that uniquely identifies the cancer care navigator;
the adjustment proposal proposition identifier; and
the proposal text; and
communicating the data representing the navigator role adjustment proposal to a visual display at a location of an administrator, the cancer care navigator, or both, wherein the role of the cancer care navigator is subsequently adjusted in delivering one or more navigation steps based on some portion of the communicated data representing the navigator role adjustment proposal.

2. (canceled)

3. The method of claim 1, wherein the evaluation value comprises a numerical answer to a proposition corresponding to the navigation steps.

4. The method of claim 1, wherein the evaluation value comprises a number selected from 1, 2, 3, 4, or 5, in which 1 means “strongly agree,” 2 means “agree,” 3 means “no opinion or neutral,” 4 means “disagree” and 5 means “strongly disagree.”

5. (canceled)

6. The method of claim 1, wherein the evaluation value may correspond to how the patient subjectively views the one or more navigation steps performed by the navigator for the patient.

7. (canceled)

8. (canceled)

9. (canceled)

10. The method of claim 1, further comprising communicating the data representing the navigator role adjustment proposal to an administrator of the patient-level, facility-level, or community-level barrier assessments, triage, resourcing, or guidance, wherein (i) any data representing the navigator role adjustment proposal regarding delivery of patient-level, barrier assessments, triage, resourcing, or guidance is communicated to a patient-level administrator, (ii) any data representing the navigator role adjustment proposal regarding delivery of facility-level barrier assessments, triage, resourcing, or guidance is communicated to a facility-level administrator, and (iii) any data representing the navigator role adjustment proposal regarding delivery of community-level barrier assessments, triage, resourcing, or guidance is communicated to a community-level administrator.

11. A method in a computing system having one or more programmable processors communicatively coupled to memory and a database on a computer readable medium for adjusting a role of a cancer care navigator, comprising:
receiving over a computer network from an input device data representing a response to an evaluation request, the data representing the response comprising:
a navigation step identifier that uniquely identifies a navigation step of cancer care delivered by the cancer care navigator to a patient, wherein the navigation step is selected from the group consisting of one or more barrier assessments, triaging, resourcing, and guidance performed for the patient;
a proposition identifier that uniquely identifies a proposition; and
an evaluation value selected from a set of evaluation values and by a person with knowledge of the navigation step of cancer care;
storing in an evaluation response data structure in the database:
the navigation step identifier in an evaluation navigation step identifier field;
the proposition identifier in an evaluation proposition identifier field; and
the evaluation value in an evaluation value field;
retrieving data representing a stored response from data representing a set of one or more stored responses from the evaluation response data structure in the database, wherein the data representing the stored response comprises:
the navigation step identifier in the evaluation navigation step identifier field;
the proposition identifier in the evaluation proposition identifier field; and
the evaluation value in the evaluation value field;
retrieving data representing a benchmark proposition from data representing a set of one or more benchmark propositions from a benchmark proposition data structure in the database, wherein the data representing the proposition benchmark comprises:
a benchmark proposition identifier in a benchmark proposition identifier field, where the benchmark proposition identifier and the proposition identifier of the stored response are equal; and
a benchmark evaluation value in a benchmark evaluation value field;
calculating an evaluation delta, wherein the evaluation delta is equal to the difference between the evaluation value of the stored response and the benchmark evaluation value;
retrieving data representing a proposition threshold from data representing a set of one or more proposition thresholds from a proposition threshold data structure in the database, wherein the data representing the proposition threshold comprises:
a proposition identifier in a proposition identifier field; and
a threshold in the threshold field;
assessing whether the evaluation delta exceeds the threshold;
retrieving data representing an adjustment proposal from data representing a set of one or more adjustment proposals from an adjustment proposal data structure in the database, wherein the data representing the adjustment proposal comprises:
an adjustment proposal proposition identifier in an adjustment proposal proposition identifier field, where the adjustment proposal proposition identifier and the threshold proposition identifier are equal; and
proposal text in a proposal field, the proposal text reciting one or more adjustments to the role of the cancer care navigator in delivering one or more navigation steps of cancer care; and
generating data representing a navigator role adjustment proposal, wherein data representing the navigator role adjustment proposal comprises:
one or more navigator identifiers that uniquely identifies the cancer care navigator;
the adjustment proposal proposition identifier; and
the proposal text; and
communicating the data representing the navigator role adjustment proposal to a visual display at a location of an administrator, the cancer care navigator, or both, wherein the role of the cancer care navigator is subsequently adjusted in delivering one or more navigation steps based on some portion of the communicated data representing the navigator role adjustment proposal.

12. (canceled)

13. (canceled)

14. The method of claim 11, wherein the evaluation value comprises a numerical answer to a proposition corresponding to the navigation steps.

15. The method of claim 11, wherein the evaluation value comprises numbers selected from 1, 2, 3, 4, or 5, in which 1 means “strongly agree,” 2 means “agree,” 3 means “no opinion or neutral,” 4 means “disagree” and 5 means “strongly disagree.”

16. The method of claim 11, wherein the evaluation value may correspond to how the patient subjectively views the one or more navigation steps performed by the navigator for the patient.

17. (canceled)

18. (canceled)

19. (canceled)

20. The method of claim 11, further comprising communicating the data representing the navigator role adjustment proposal to an administrator of the patient-level, facility-level, or community-level barrier assessments, triage, resourcing, or guidance, wherein (i) any data representing the navigator role adjustment proposal regarding delivery of patient-level, barrier assessments, triage, resourcing, or guidance is communicated to a patient-level administrator, (ii) any data representing the navigator role adjustment proposal regarding delivery of facility-level barrier assessments, triage, resourcing, or guidance is communicated to a facility-level administrator, and (iii) any data representing the navigator role adjustment proposal regarding delivery of community-level barrier assessments, triage, resourcing, or guidance is communicated to a community-level administrator.
